SEQUENCE LISTING (6 sequences)

SEQ ID NO: 1 - DNA, 2034 bp, Homo sapiens
atgccggggttcctggttcgcatcctccttctgctgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatga

SEQ ID NO: 2 - DNA, 1751 bp, Artificial Sequence
  CAG Promoter
tgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccacc

SEQ ID NO: 3 - DNA, 510 bp, Homo sapiens
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 4 - DNA, 130 bp, Artificial Sequence
  Left Inverted Terminal Repeat Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 5 - DNA, 141 bp, Artificial Sequence
  Right Inverted Terminal Repeat Sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 4600 bp, Artificial Sequence
  LYS-GM101 Vector Construct
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccaccatgccggggttcctggttcgcatcctccttctgctgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2021/0355506 A1
(43) Pub. Date: Nov. 18, 2021

(54) COMPOSITIONS AND METHODS FOR TREATING GM1 GANGLIOSIDOSIS AND OTHER DISORDERS

(71) Applicant: LYSOGENE, Neuilly sur Seine (FR)

(72) Inventors: Michaël HOCQUEMILLER, Neuilly sur Seine (FR); Karen PIGNET-AIACH, Neuilly sur Seine (FR); Ralph LAUFER, Neuilly sur Seine (FR); Sophie OLIVIER, Neuilly sur Seine (FR); Samantha PARKER, Neuilly sur Seine (FR)

(21) Appl. No.: 17/319,866

(22) Filed: May 13, 2021

Related U.S. Application Data
(60) Provisional application No. 63/024,298, filed on May 13, 2020.

(51) Int. Cl. C12N 15/86; A61K 48/00; A61K 31/436; A61K 31/5377; A61K 31/573; A61K 38/47; A61P 25/28; C12N 7/00; A61K 9/00

(52) U.S. Cl. CPC C12N 15/86; A61K 48/0066; A61K 48/0075; A61K 31/436; A61K 31/5377; A61K 31/573; C12N 2750/14171; C12Y 302/01023; A61P 25/28; C12N 7/00; A61K 9/0085; C12N 2750/14121; C12N 2750/14143; A61K 38/47

(57) ABSTRACT

The disclosure provides gene therapy vectors and methods of use thereof for treating genetic diseases, such as lysosomal storage diseases. For example, the disclosure provides gene therapy vectors and methods for treating GM1 gangliosidosis. The disclosure also provide methods for making the provided gene therapy vectors.

### CROSS REFERENCE TO RELATED APPLICATIONS

This application claims the benefit of priority to U.S. Provisional Application No. 63/024,298, filed on May 13, 2020, the entire contents of which are hereby incorporated by reference.

### SEQUENCE LISTING

The Sequence Listing associated with this application is provided in text format in lieu of a paper copy, and is hereby incorporated by reference into the specification. The name of the text file containing the Sequence Listing is LYSO-004_01US SeqList_ST25.txt. The text file is 13 KB, was created on May 13, 2021, and is being submitted electronically via EFS-Web.

### BACKGROUND

GM1 gangliosidosis is a severe debilitating and life-threatening lysosomal storage disease (LSD) affecting children. GM1 gangliosidosis is caused by mutations in the GLB1 gene encoding the lysosomal acid beta-galactosidase (β-gal) enzyme. The resulting enzyme deficiency leads to accumulation of GM1 ganglioside in neurons and progressive neurodegeneration. Children affected by GM1-gangliosidosis suffer from severe and eventually lethal motor and developmental defects. Type I (infantile) GM1 gangliosidosis occurs in infants with an onset before 6 months of age and a life expectancy of about 3 years. For type IIa (late-infantile) GM1 gangliosidosis, onset occurs between infancy and 2 years of age, with a life expectancy of less than 10 years. For Type IIb (juvenile) GM1 gangliosidosis onset occurs during childhood, with a life expectancy of less than 30 years. Type III (adult) GM1 gangliosidosis occurs in early adulthood, and survival is variable.

There is currently no treatment available for patients with GM1 gangliosidosis. Only supportive treatment can be offered in this fatal disease. Supportive treatment includes adequate nutrition to maintain growth, speech therapy, seizure control, routine management of risk of aspiration and hospice services for supportive in-home care. Important attention must also be paid to the prevention of complications, via routine immunization and bacterial endocarditis prophylaxis in patients with cardiac valvular involvement, and anesthetic precautions when there is a skeletal involvement and where airways are compromised (Regier and Tifft 2013).

Thus, there is an urgent need for effective therapies for LSD such as GM1 gangliosidosis. The present disclosure addresses this and other needs.

### BRIEF SUMMARY OF THE DISCLOSURE

The present disclosure provides gene therapy vectors and methods of use thereof for treating lysosomal storage disorders such as GM1 gangliosidosis. In embodiments, the present disclosure provides methods for treating lysosomal storage disorders such as GM1 gangliosidosis by administering a gene therapy vector or composition comprising a gene therapy vector encoding a human β-gal or an active variant thereof, wherein the vector or composition is administered to the cerebrospinal fluid (CSF) of a subject. In embodiments, the present disclosure provides methods for treating lysosomal storage disorders such as GM1 gangliosidosis by administering a gene therapy vector or composition comprising a gene therapy vector encoding a human β-gal or an active variant thereof, wherein the vector or composition is administered to a subject via intra-cisterna magna (ICM) injection.

In embodiments, the present disclosure provides a replication deficient adeno-associated virus serotype rh.10 (AAVrh.10)-derived vector comprising an expression cassette comprising in the following 5′ to 3′ order: a promoter sequence; a polynucleotide sequence encoding a human β-gal or an active variant thereof; and a polyadenylation (polyA) sequence. In embodiments, the promoter sequence is derived from a CMV early enhancer/chicken beta actin (CAG) promoter sequence. In embodiments, the polyA sequence is derived from a human growth hormone 1 sequence.

In embodiments, the present disclosure provides a replication deficient AAVrh.10-derived vector comprising an expression cassette, wherein the expression cassette consists of, in the following 5′ to 3′ order: a promoter sequence derived from a CAG promoter sequence; a polynucleotide sequence encoding a human β-gal or an active variant thereof; and a polyA sequence derived from a human growth hormone 1 polyA sequence.

In embodiments, the expression cassette provided herein is flanked by two AAV2 internal terminal repeat (ITR) sequences, wherein one of the two AAV2 ITR sequences is located 5′ of the expression cassette and one of the two AAV2 ITR sequences is located 3′ of the expression cassette. In embodiments, the ITR sequence located at the 5′ end of the expression cassette comprises the nucleotide sequence according to SEQ ID NO: 4 and the ITR sequence located at the 3′ end of the expression cassette comprises the nucleotide sequence according to SEQ ID NO:

In embodiments, the vector provided herein comprises a polynucleotide sequence encoding a human β-gal, wherein the polynucleotide comprises the sequence according to SEQ ID NO: 1. In embodiments, CAG promoter sequence provided herein comprises the sequence according to SEQ ID NO: 2. In embodiments, the polyadenylation (polyA) sequence comprises the sequence according to SEQ ID NO: 3.

In embodiments, the present disclosure provides a replication deficient AAVrh.10-derived vector comprising an expression cassette, wherein the expression cassette comprises, in the following 5′ to 3′order: an AAV2 ITR sequence; a promoter sequence derived from a CAG promoter sequence; a polynucleotide sequence encoding a human β-gal or an active variant thereof; a polyA sequence derived from a human growth hormone 1 polyA sequence; and an AAV ITR sequence. In embodiments, the vector comprises the sequence according to SEQ ID NO: 6.

In embodiments, the present disclosure provides compositions comprising the vectors provided herein, and a pharmaceutically acceptable carrier. In embodiments, the compositions provided herein comprise the vector at a concentration of about 1.0E+12 vg/mL to about 5.0E+13 vg/mL. In embodiments, the concentration of the vector in the composition is about 1.8E+13 vg/mL.

In embodiments, the present disclosure provides methods for treating lysosomal storage disorders, such as GM1 gangliosidosis. In embodiments, the methods comprise administering a vector provided herein or a composition provided herein to a subject in need thereof In embodiments, the disclosure provides a vector provided herein for use as a medicament for the treatment of GM1 gangliosidosis. In embodiments, the disclosure provides a composition provided herein for use as a medicament for the treatment of GM1 gangliosidosis. In embodiments, the methods and uses provided herein comprise administration of the vectors or compositions provided herein to the cerebrospinal fluid (CSF) of the subject in need thereof. In embodiments, the methods and uses provided herein comprise administration of the vectors or compositions provided herein to the subject in need thereof via intra-cisterna magna (ICM) injection. In embodiments, the vectors and compositions are formulated for administration to the CSF. In embodiments, the vectors and compositions are formulated for administration via ICM injection. In embodiments, the vectors and compositions provided herein are for administration to the CSF of the subject. In embodiments, the vectors and composition provided herein are for administration via ICM injection. In embodiments, the vectors and compositions provided herein are administered to the subject in a volume of about 0.1 mL/kg body weight to about 1.0 mL/kg body weight. In embodiments, the vectors and compositions provided herein are administered to the subject in a volume of about 0.8 mL/kg body weight. In embodiments, the vectors and compositions provided herein are administered to the subject in a volume of about 0.4 mL/kg body weight. In embodiments, the vectors and compositions provided herein are administered to the subject in a volume of about 1 mL to about 15 mL, e.g., in a volume of about 2 mL to about 12 mL, e.g., in a volume of about 2 mL to 6 mL. In embodiments, a volume of cerebrospinal fluid (CSF) is removed prior to administration of the vector or composition. For example, in embodiments, the volume of CSF that is removed prior to administration of the vector or composition corresponds to about half of the volume of the vector or composition to be administered. In other embodiments, the volume of CSF that is removed prior to administration of the vector or composition corresponds to the volume of the vector or composition to be administered

In embodiments, the methods and uses provided herein comprise administration of a dose of between about 1.0E+12 vg/kg body weight to about 1.0E+13 vg/kg body weight of the vector to the subject in need thereof. In embodiments, the dose of the vector is about 7.2E+12 vg/kg body weight. In embodiments, the dose of the vector is calculated based on the expected or approximate volume of CSF in the subject. For example, in embodiments, the dose of the vector administered is from about 5.0E+11 vg/mL of CSF to about 5.0E+12 vg/mL of CSF. In embodiments, the dose of the vector of about 1.8E+12 vg/mL of CSF. In embodiments, the total dose of the vector is about 1.0E+13 vg to about 5.0E+14 vg, or about 4E+13 vg to about 1.2E+14 vg.

In embodiments, the methods and uses provided herein further comprise administering an immunosuppressive regimen to the subject. In embodiments, the immunosuppressive regimen comprises tacrolimus, mycophenolate mofetil, and/or prednisone.

In embodiments, the present disclosure provides kits comprising a LYS-GM101 vector provided herein and instructions for use thereof.

### BRIEF DESCRIPTION OF THE DRAWINGS

FIG. 1A is a schematic representation of an Adeno Associated Virus vector construct, LYS-GM101. LYS-GM101 is an adeno-associated virus (AAV) serotype rh.10 expressing human beta-galactosidase (AAVrh.10-CAG-βgal). FIG. 1B and FIG. 1C provide the full vector sequence (SEQ ID NO: 6).

FIGS. 2A-2F shows the β-gal enzyme activity and GM1 ganglioside levels in the brain, cerebellum, and spinal cord at 1 month after injection of AAVrh.10-mβgal. AAVrh.10-mβgal was injected bilaterally in thalamus (2×2.22 μl) or cerebral lateral ventricle (14.8 μl). Mice (n=4-6) per group were euthanized at 1 month post-injection and β-gal activity (FIG. 2A, FIG. 2B & FIG. 2C) and GM1 ganglioside storage (FIG. 2D, FIG. 2E & FIG. 2F), measured in brain (FIG. 2A & FIG>2D), cerebellum (FIG. 2B & FIG. 2E) and spinal cord (FIG. 2C & FIG. 2F). *p<0.05 compared to PBS (GM1 gangliosidosis animals injected with PBS via Thal and ICV combined). Blue line corresponds to normal levels assessed from non-injected WT mice.

FIG. 3 shows the spatial distribution of β-gal enzyme at 1 month. Distribution of enzyme was assessed by histochemical staining with X-gal at low pH (blue stain) in sagittal sections of brain. Thal: Thalamic injection; ICV: Intracerebroventricular injection. NA: Not applicable.

FIG. 4 shows the brain and spinal cord regions for assessing GM1 gangliosidosis in the cat study. At necropsy, the brain was cut into 6 mm blocks from the frontal pole through caudal cerebellum, for a total of 9 blocks (A-I). From each block, the right hemisphere was frozen in OCT media for enzyme assays, and the left hemisphere was further cut in half and stored in 10% formalin (rostral half) or frozen in liquid nitrogen and stored at −80° C. (caudal half). The spinal cord was removed in its entirety, and 7 regions were assayed (J-P). The spinal cord was stored in OCT or 10% formalin, or frozen in liquid nitrogen for storage at −80° C.

FIG. 5 shows the β-gal enzyme activity in the CNS of GM1 gangliosidosis cat at 1 month. β-gal activity was analyzed in the CNS blocks described in FIG. 8 (brain A to I; spinal cord J to P) and expressed as ‘fold of normal’ activity, meaning that β-gal enzyme activity in each CNS block from treated animals was standardized to levels in the corresponding block from normal animals (n=3). Statistical significance was determined using a 2-tailed t-test. Symbols denote p<0.05 compared to the following groups: untreated GM1 gangliosidosis cat (+); lumbar cistern

FIG. 6 shows filipin staining of storage material in the GM1 gangliosidosis cat CNS at 1 month. Filipin staining appears as punctate white or gray dots in gray matter of untreated GM1 gangliosidosis cats, with little staining in gray matter of WT cats. Filipin staining was apparent in the cerebrum (block D located in FIG. 8) of all AAV-treated cats, with moderately diminished staining in the cat treated by intra-cisterna magna (ICM) injection. Cerebellar gray matter and brainstem (block H located in FIG. 8) exhibited profound clearance of storage material after CM injection, but little clearance after bilateral ICV or ITL infusions. Filipin staining was reduced in the lumbar intumescence of the spinal cord (block P located in FIG. 8) of all treated cats.

FIG. 7 shows the disease progression of individual untreated and treated GM1 gangliosidosis cats. Data points are accompanied by a trend line for the average score. Also shown is the average score of WT cats

FIG. 8 shows the biomarkers of neurodegeneration in the cat study. AST and LDH levels in CSF samples collected at the humane endpoint of untreated or treated GM1 gangliosidosis cats (8 months or 11 months, respectively). *p<0.05 v. normal, age-matched cats (n=5); +p<0.05 v. untreated GM1 gangliosidosis cats (n=5).

FIG. 9 shows the biodistribution of β-gal in the CNS in the cat study. Brain and spinal cord samples collected as described in FIG. 8 (brain A to I; spinal cord J to P) were stained with Xgal, which forms a blue precipitate when cleaved by β-gal. Shown on left panel for comparison are untreated normal and GM1 controls (brain section E and spinal section L). White matter of untreated GM1 cats consistently shows background staining.

FIG. 10 shows β-gal activity levels in the CNS in the cat study. β-gal activity was analyzed in the CNS blocks described in FIG. 8 (brain A to I; spinal cord J to P) and expressed as ‘fold of normal’ activity, meaning that β-gal enzyme activity in each CNS block from treated animals was standardized to levels in the corresponding block from normal animals (n=5). Dashed horizontal line represents normal activity. Statistical significance was determined using a 2-tailed t-test. * denote p<0.05 compared to normal

FIG. 11 is an illustration of β-gal activity distribution in the NHP brain at 12 weeks. Examples of even brain slabs divided into 10×10 mm sections from one Group 1 animal (M191888 left panel) and one Group 3 animal (F191907 right panel). β-gal enzyme activity values, expressed in nmol of 4-MU/h/mg of protein, of each 10×10 mm sections are presented in combination with a color code ranging from light orange (lowest β-gal enzyme activity) to dark orange (highest β-gal enzyme activity).

FIG. 12 shows the mean β-gal activity in NHP CNS at 12 weeks. Mean values of β-gal enzyme activity in the brain and spinal cord of NHP expressed in nmol of 4-MU/h/mg of protein. Statistical significance was determined using a 2-tailed t-test. * denote p<0.001 compared to Group 1.

### DETAILED DESCRIPTION

In embodiments, the present disclosure provides novel compositions and methods useful in treating a variety of diseases and disorders, including genetic diseases (including those resulting from a gene deletion or mutation leading to reduced expression or lack of expression of an encoded gene product, the expression of an altered form of a gene product, or disruption of a regulatory element controlling the expression of a gene product), neurological diseases and disorders, and diseases and disorders of the brain. In embodiments, the disclosure relates to gene therapy for lysosomal storage disorders, such as GM1 gangliosidosis. In embodiments, the gene therapy for lysosomal storage disorders such as GM1 gangliosidosis is administered to the cerebrospinal fluid (CSF) of a subject. In embodiments, the gene therapy for lysosomal storage disorders such as GM1 gangliosidosis is administered to a subject via intra-cisterna magna (ICM) injection. In embodiments, the gene therapy comprises a gene therapy vector or a composition comprising a gene therapy vector, encoding a human β-gal or an active variant thereof.

GM1 gangliosidosis is an autosomal recessive disease caused by mutations in the GLB1 gene encoding for the lysosomal acid β-galactosidase enzyme (β-gal). β-gal hydrolyses terminal galactose residues of galactose containing oligosaccharides, keratan sulfate, and other β-galactose-containing glycoconjugates. Its reduced or null activity in cells, caused by mutations in the GLB1 gene, leads to substrate (GM1 ganglioside and its asialo derivate GA1) accumulation to toxic levels in many tissues, particularly the brain, resulting in progressive neurodegeneration, cognitive and motor defects, seizures, and premature death. There are currently no approved and/or effective treatments. The disease is always fatal in children. In addition to the predominant brain and spinal cord pathology, multiple other organs are affected. Further pathologies include visual deficits, bone/skeletal dysfunction and hepatosplenomegaly.

Classification of GM1 gangliosidosis is as follows. Type I (infantile) is characterized by onset at less than 6 months of age and death at about 3 years; incidence is about 1:250,000-1:300,000. Type IIa (late infantile) is characterized by onset at 12-24 months of age and death in the first decade; incidence is about 1:500,000. Type IIb (juvenile) is characterized by onset at 4-6 years of age, and survival into the 3r^{d} decade; incidence is about 1:500,000. Type III (adult) is characterized by onset in early adulthood, with variable survival; incidence is unknown. Disease severity generally decreases with age of onset.

Bone marrow transplantation was not successful in treating the neurological complications in case reports of juvenile GM1 gangliosidosis (Shield, Stone, and Steward 2005). Miglustat combined with ketogenic diet is under clinical investigation. Preliminary results in early infantile GM1 gangliosidosis suggest positive impact on life expectancy, but no impact on motor or cognitive functions (James Utz et al. 2017). Substrate reduction using imino sugars successfully inhibited ganglioside biosynthesis and reduced accumulation in rodent CNS (Kasperzyk et al. 2005) but it is not known whether this approach has therapeutic benefit in patients. A chemical chaperone (N-octyl-4-epi-β-valienamine, NOEV) that stabilizes the enzyme was shown to lead to increased β-gal activity in mice with prevention of neurological deterioration (Matsuda et al. 2003). This therapy is however dependent on subjects having residual β-gal activity. Deep brain stimulation in a patient with adult-onset GM1 gangliosidosis showed functional improvement of dystonia but no change in disease progression. Finally, AAV-based delivery of the GLB1 gene in GM1 gangliosidosis mice or cats has shown to result in sustained correction of the disease phenotype (McCurdy et al. 2014); (Weismann et al. 2015); (Hayward et al. 2015); (Regier et al. 2016). However, the major challenge in treating lysosomal storage diseases by AAV gene therapy is to achieve widespread therapeutic levels of the deficient enzyme throughout all affected tissues, in particular the brain and the spinal cord.

Different routes of CNS delivery were investigated in the studies provided herein, including intra-cranial injections (into the thalamus and deep cerebellar nuclei [DCN] or intracerebroventricular [ICV] injections) in GM1 gangliosidosis mice, and intracisternal infusions (ICV, ICM or intrathecal lumbar [ITL]) in the GM1 gangliosidosis cat model. Injection of a gene therapy vector provided herein into the cisterna magna of nonhuman primates (NHP) at doses similar to the intended human clinical doses led to significant elevations of β-gal activity throughout the brain and spinal cord relative to non-injected controls at 12 weeks post-administration. Accordingly, the present disclosure demonstrates that intra-cerebrospinal fluid (CSF) delivery, e.g. via intracisternal injection (ICM), is the optimal route of administration for the treatment of GM1 gangliosidosis, for example, via the GM101 therapy provided herein.

For example, in embodiments, the present disclosure provides GM101 (also referred to herein as “LYS-GM101”), which is a replication-defective recombinant adeno-associated virus rh.10 (AAVrh.10) vector engineered to carry the therapeutic gene of interest, GLB1. The vector is comprised of an expression cassette including a CAG promoter, the GLB1 cDNA, and the human growth hormone poly A sequence, flanked by AAV2 inverted terminal repeats (ITR), packaged inside the AAVrh.10 protein shell (capsid). The therapeutic goal of LYS-GM101 gene therapy is to restore long-term expression of β-gal in the central nervous system (CNS), including the brain and spinal cord, thereby removing accumulated GM1 ganglioside and asialo GM1 (GA1), and preventing the de novo accumulation of GM1 ganglioside.

Accordingly, in embodiments, the present disclosure provides methods for achieving widespread therapeutic levels of the deficient enzyme throughout all affected tissues in GM1 gangliosidosis patients. In embodiments, the methods involve intra-CSF delivery of an AAV-vectored GLB1 gene therapy to subjects in need thereof

Without wishing to be bound by theory, upon injection into the cisterna magna, the AAV vector particle diffuses locally, attaches to cell surface receptors, and may also be transported along axons or interstitial fluid to remote anatomical CNS structures. The vector particles are internalized by neuronal or glial cells. Each of these cell types are deficient for the β-gal enzyme in GM1 gangliosidosis patients and suffer from the toxic accumulation of gangliosides substrates. Upon entry into the cells, the recombinant genome encoding the β-gal protein is transported into the nucleus where it undergoes a series of molecular transformations that result in its stable establishment as a double stranded deoxyribonucleic acid (DNA) molecule. This DNA is transcribed into messenger ribonucleic acids (mRNAs) by the cellular machinery. The mRNAs are translated into the protein β-gal, which will restore the cellular enzyme deficiency.

Enzyme complementation and correction of lysosomal storage occurs by three different mechanisms. 1) The enzyme may reach the lysosome of cells which contain and express the AAV-borne transgene and degrade the accumulated catabolites. 2) The enzyme made within the genetically modified cells may be released from these cells, recaptured by adjacent cells, and rerouted toward their lysosomes. This phenomenon is known as “cross-correction” (Tomanin et al., 2012). In embodiments, following cell transduction by AAV and enzyme expression, lyosomal enzyme can be secreted and cross-correct neighboring cells via mannose-6-phosphate receptor-mediated uptake. 3) Anterograde and retrograde transport of AAV vectors or the secretable enzyme can result in transport of the therapeutic enzyme to sites distant from the injection site (Chen et al., 2006).

As will be appreciated by one of skill in the art, while certain compositions and methods are specifically exemplified herein, the present disclosure is not so limited but includes additional embodiments and uses, including, but not limited to, those specifically described herein. In addition, in the following description, certain specific details are set forth in order to provide a thorough understanding of various embodiments of the disclosure. However, one skilled in the art will understand that the disclosure may be practiced without these details.

### Definitions

Unless defined otherwise, all technical and scientific terms used herein have the same meaning as commonly understood by those of ordinary skill in the art to which the invention belongs. For the purposes of the present disclosure, the following terms are defined below.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

The term “active variant” indicates and encompasses both “biologically active fragments” and “biologically active variants.” Representative biologically active fragments and biologically active variants generally participate in an interaction, e.g., an intra-molecular or an inter-molecular interaction. An inter-molecular interaction can be a specific binding interaction or an enzymatic interaction. Examples of enzymatic interactions or activities include, without limitation, dehydroxylation and other enzymatic activities described herein.

The term “biologically active fragment”, as applied to fragments of a reference polynucleotide or polypeptide sequence, refers to a fragment that has at least about 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of at least one activity (e.g., an enzymatic activity) of a reference sequence. The term “reference sequence” refers generally to a nucleic acid coding sequence or amino acid sequence to which another sequence is being compared. All sequences provided in the Sequence Listing are also included as reference sequences. Included within the scope of the present disclosure are biologically active fragments of at least about 10, 11, 12, 13, 14, 15, 16, 17, 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600 or more contiguous nucleotides or amino acid residues in length, including all integers in between.

The term “biologically active variant”, as applied to variants of a reference polynucleotide or polypeptide sequence, refers to a variant that has at least about 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of an activity (e.g., an enzymatic activity) of a reference sequence. Included within the scope of the present disclosure are biologically active variants having at least about 50%, at least about 60%, at least about 70%, at least about 80% at least about 90%, at least about 95%, at least about 98%, or at least about 99% identity with a reference sequence, including all integers in between.

By “coding sequence” is meant any polynucleotide sequence that contributes to the code for the polypeptide product of a gene. By contrast, the term “non-coding sequence” refers to any polynucleotide sequence that does not contribute to the code for the polypeptide product of a gene.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the terms “function” and “functional”, and the like, refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

The recitations “mutation” or “deletion,” in relation to a gene refer generally to those changes or alterations in a gene that result in decreased or no expression of the encoded gene product or that render the product of the gene non-functional or having reduced function as compared to the wild-type gene product. Examples of such changes include nucleotide substitutions, deletions, or additions to the coding or regulatory sequences of a target gene, in whole or in part, which disrupt, eliminate, down-regulate, or significantly reduce the expression of the polypeptide encoded by that gene, whether at the level of transcription or translation, and/or which produce a relatively inactive (e.g., mutated or truncated) or unstable polypeptide. In certain aspects, a targeted gene may be rendered “non-functional” by changes or mutations at the nucleotide level that alter the amino acid sequence of the encoded polypeptide, such that the modified polypeptide is expressed, but has reduced function or activity with respect to one or more enzymatic activity, whether by modifying that polypeptide' s active site, its cellular localization, its stability, or other functional features apparent to a person skilled in the art.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a polynucleotide or polypeptide is isolated from, or derived from, a particular source, such as a desired organism or a specific tissue within a desired organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition. In the absence of that condition, inducible promoters typically do not allow significant or measurable levels of transcriptional activity. Numerous standard inducible promoters will be known to one of skill in the art.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes both single and double stranded forms of DNA and RNA.

The term “polynucleotide variant” refers to polynucleotides displaying substantial sequence identity with a reference polynucleotide sequence or polynucleotides that hybridize with a reference sequence under stringent conditions that are defined hereinafter. This term also encompass polynucleotides that are distinguished from a reference polynucleotide by the addition, deletion or substitution of at least one nucleotide. Accordingly, the term “polynucleotide variant” includes polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

With regard to polynucleotides and polypeptides, the term “exogenous” refers to a polynucleotide or polypeptide sequence that does not naturally occur in a wild-type cell or organism, but is typically introduced into the cell by molecular biological techniques. Examples of exogenous polynucleotides include vectors, plasmids, and/or man-made nucleic acid constructs encoding a desired protein. With regard to polynucleotides and polypeptides, the term “endogenous” or “native” refers to naturally-occurring polynucleotide or polypeptide sequences that may be found in a given wild-type cell or organism.

An “introduced” polynucleotide sequence refers to a polynucleotide sequence that is added or introduced into a cell or organism. The “introduced” polynucleotide sequence may be a polynucleotide sequence that is exogenous to the cell or organism, or it may be a polynucleotide sequence that is already present in the cell or organism. For example, a polynucleotide can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation “polypeptide variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues. Included are polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing). In particular embodiments, the polypeptide variant maintains at least one biological activity of the reference polypeptide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Transformation” refers to the permanent, heritable alteration in a cell resulting from the uptake and incorporation of foreign DNA into the host-cell genome or maintained extrachromosomally within the host cell; also, the transfer of an exogenous gene from one organism into the genome of another organism.

As used herein, the terms “treatment,” “treat,” “treated” or “treating” refer to prophylaxis and/or therapy, particularly wherein the object is to prevent or slow down (lessen) an undesired physiological change or disorder, such as the development and/or progression of a brain disorder resulting from a mutated gene, such as, e.g., a lysosomal storage disease (LSDs). Beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” can also mean prolonging survival and/or increased quality of life as compared to expected survival and/or quality of life if not receiving treatment. Those in need of treatment include those already with the condition or disorder (e.g., brain disorder resulting from a mutated gene, such as GM1 gangliosidosis) as well as those prone to have the condition or disorder or those in which the condition or disorder is to be prevented. Thus, “treatment” also includes administration of the compounds of the disclosure to those individuals thought to be predisposed to the disease due to familial history, genetic or chromosomal abnormalities, and/or due to the presence of one or more biological markers for the disease, e.g., to inhibit, prevent, or delay onset of the disease, or reduce the likelihood of occurrence of the disease. In particular embodiments, treatment may include any of the following: decrease of developmentally regression, decrease of language impairment or improvement of language development, decrease of motor skill impairment, decrease of intellectual development impairment, decrease of hyperactivity (excess motor activity), improvement in sleep, attention, decrease of physical and mental ability impairment (patients lose complete motor abilities (walking, speech, feeding, etc.), cognitive abilities, severe seizures, decrease of impairment, such as airway obstruction and cardiac failure. In embodiments, “treatment” includes making the cells able to produce the missing enzyme treating and/or reversing the consequences of the disease, e.g., restoring or providing the function of the GLB1 gene to a subject, or breaking down the accumulated GM1 ganglioside and asialo GM1 (GA1).

A “subject” includes a mammal, e.g., a human, including a mammal in need of treatment for a disease or disorder, such as a mammal having been diagnosed with having a disease or disorder or determined to be at risk of developing a disease or disorder. In particular examples, a subject is a mammal diagnosed with a genetic disease, a brain disorder, or a neurological disease or disorder, such as a lysosomal storage disorder, including GM1 gangliosidosis. In embodiments, the subject is a human, and may be and adult or a non-adult. In embodiments, the subject is a child or an infant.

By “vector” is meant a polynucleotide molecule, e.g., a DNA molecule derived, for example, from a plasmid, bacteriophage, yeast or virus, into which a polynucleotide can be inserted or cloned. A vector typically contains one or more unique restriction sites and can be capable of autonomous replication in a defined host cell, or be integrable with the genome of the defined host such that the cloned sequence is reproducible. Accordingly, a vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. A vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. “Vectors” also include viruses and viral particles into which a polynucleotide can be inserted or cloned. Such may be referred to as “viral vectors.” “Gene therapy vectors” are vectors, including viral vectors, used to deliver a therapeutic polynucleotide or polypeptide sequence to a subject in need thereof, which is typically a polynucleotide or polypeptide sequence missing, mutated or having deregulated expression in the subject, e.g., due to a genetic mutation in the subj ect.

A common means to insert a DNA sequence of interest into a DNA vector involves the use of enzymes called restriction enzymes that cleave DNA at specific sites called restriction sites. A “cassette” or “gene cassette” or “expression cassette” refers to a polynucleotide sequence that encodes for one or more expression products, and contains the necessary cis-acting elements for expression of these products, that can be inserted into a vector at defined restriction sites.

The term “wild-type”, as used herein, refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

### Gene Therapy Vectors

In certain embodiments, the present disclosure includes gene therapy vectors for the treatment of GM1 gangliosidosis. Such gene therapy vectors may be used to deliver a human β-gal or an active variant thereof to a cell within a subject in need thereof. As described in the accompanying examples, studies have established that the gene therapy vectors of the present disclosure are both efficacious and safe for the treatment of GM1 gangliosidosis. In embodiments, the studies provided in the accompanying examples establish dosing routes and/or doses and/or dosing regimens that provide superior effects in the treatment of GM1 gangliosidosis patients.

Without wishing to be bound by theory, it is understood that upon administration, the gene therapy vector particles provided herein, and the enzymes produced, will diffuse locally, as well as be transported along axons to remote anatomical CNS structures to allow for the correction of extended CNS regions. Upon entry into cells, the gene therapy vector comprising GLB1 (the gene encoding β-gal) will be transported into the nucleus where it will undergo a series of molecular transformations resulting in the stable establishment as a double stranded deoxyribonucleic acid (DNA) molecule. This DNA will be transcribed into messenger ribonucleic acids (mRNAs), which in turn will translate into β-gal, the missing enzyme in GM1 gangliosidosis patients. Transduced cells will express and deliver the enzyme continuously, thus constituting a permanent CNS source of enzyme production to complement the lacking endogenous enzyme. The gene therapy vector described herein is LYS-GM101, also referred to herein as GM101 or AAVrh10-GM101. LYS-GM101 comprises a replication deficient adeno-associated virus serotype rh.10 (AAVrh.10) comprised of a defective AAV2 genome containing the GLB1 gene. In addition, the present disclosure provides an improved delivery system for LYS-GM101 that provides superior gene expression throughout the brain and spinal cord. In embodiments, LYS-GM101 is administered via a ICM injection route. Such an injection route coupled with the compositions and methods provided herein result in broad brain distribution of the enzyme and enhanced efficacy in treating GM1 gangliosidosis.

It was discovered via the studies provided herein that the gene therapy vectors of the present disclosure provide unexpected advantages over those previously described, including high levels of β-gal expression in the CNS following ICM injection. In addition, the compositions and methods of the present disclosure provide enhanced efficacy via improved expression of the therapeutic product, broader distribution of expression, and more efficient delivery via optimal dosing.

Adeno-associated virus (AAV), a member of the Parvovirus family, is a small, nonpathogenic, nonenveloped, icosahedral virus with single-stranded linear DNA genomes of 4.7 kilobases (kb) to 6 kb. AAV's life cycle includes a latent phase at which AAV genomes, after infection, are site specifically integrated into host chromosomes and an infectious phase in which, following either adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer. The members of this genus require a helper virus, such as adenovirus or herpes simplex virus, to facilitate productive infection and replication. In absence of a helper virus, AAVs establish a latent infection within the cell, either by site-specific integration into the host genome (rare) or by persisting in episomal forms.

To date, at least a dozen different serotypes of AAVs with variations in their surface properties have been isolated from human or non-human primates (NHP) and characterized. The term “serotype” is a distinction with respect to an AAV having a capsid which is serologically distinct from other AAV serotypes. Serologic distinctiveness is determined on the basis of the lack of cross-reactivity between antibodies to one AAV serotype as compared to other AAV serotypes. The gene therapy vectors, also named vector, of the disclosure may have any one of the known serotypes (rh) of AVV, for example, any one of rh1, rh2, rh3, rh4, rh5, rh6, rh7, rh8, rh9 or rh10, preferably rh10. These various AAV serotypes may also be referred to as AAV1, AAV2, AAV3, AAV4, AAV5, AAV6, AAV7, AAV8, AAV9 or AAV10 (AAVrh.10).

In embodiments, vectors of the disclosure may have an artificial AAV serotype. Artificial AAV serotypes include, without limitation, AAVs with a non-naturally occurring capsid protein. Such an artificial capsid may be generated by any suitable technique, using a novel AAV sequence of the disclosure (e.g., a fragment of a vp1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

The AAV capsid is assembled from 60 viral protein (VP) subunits (VP1, VP2 and VP3). The core VP monomer (VP3) has a jellyroll, beta barrel structure comprised of 7 anti-parallel f3 strands connected by interdigitating loop regions. Portions of these highly variable loops are surface-exposed and define the topology of the AAV capsid, which, in turn, determines tissue tropism, antigenicity, and receptor usage across the various AAV serotypes.

AAV serotype rh.10 (AAVrh.10) is described in PCT Patent Application Publication No. WO 2003/042397. AAVrh.10 vectors have been shown to transduce neurons and astrocytes in the neonatal mouse central nervous system (Zhang, H., et al., Molecular Therapy 19, 1440-1448 (August 2011)). In addition, AAVrh.10 vectors has superior activity upon injection into the brain of rodents, and there is no natural disease with AAV serotype rh.10 in the human population.

The AAV genome is relatively simple, containing two open reading frames (ORFs) flanked by short inverted terminal repeats (ITRs). The ITRs contain, inter alia, cis-acting sequences required for virus replication, rescue, packaging and integration. The integration function of the ITR permits the AAV genome to integrate into a cellular chromosome after infection.

The nonstructural or replication (Rep) and the capsid (Cap) proteins are encoded by the 5′ and 3′ open reading frames (ORFs), respectively. Four related proteins are expressed from the rep gene; Rep78 and Rep68 are transcribed from the p5 promoter while a downstream promoter, p19, directs the expression of Rep52 and Rep40. Rep78 and Rep68 are directly involved in AAV replication as well as regulation of viral gene expression. The cap gene is transcribed from a third viral promoter, p40. The capsid is composed of three proteins of overlapping sequence; the smallest (VP-3) is the most abundant. Because the inverted terminal repeats are the only AAV sequences required in cis for replication, packaging, and integration, most AAV vectors dispense with the viral genes encoding the Rep and Cap proteins and contain only the foreign gene(s), e.g., therapeutic gene(s), inserted between the terminal repeats.

The GLB1 gene encodes for the lysosomal acid β gal enzyme. β galactosidase (β-gal) is the deficient enzyme involved in GM1 gangliosidosis. β-gal is an enzyme that hydrolyses terminal galactose residues of galactose containing oligosaccharides, keratan sulfate, and other β-galactose-containing glycoconjugates. Its reduced or null activity in cells, caused by mutations in the GLB1 gene, leads to substrate (GM1 ganglioside and its asialo derivate GA1) accumulation to toxic levels in many tissues, particularly the brain, resulting in progressive neurodegeneration and premature death.

In embodiments, the gene therapy vectors of the present disclosure comprise polynucleotide sequences encoding GLB 1. In embodiments, a gene therapy vector of the present disclosure is an AAV serotype rh10 vector comprising a polynucleotide sequence encoding the human GLB1 polypeptide or an active variant thereof. In embodiments, these gene therapy vectors may be administered to a subject in need thereof in a replication deficient AAVrh.10 vector comprising a defective AAV2 genome comprising a polynucleotide sequence encoding β-gal or an active variant thereof driven by a promoter and packaged in capsid of AAVrh.10.

In embodiments, the gene therapy vector further comprises additional regulatory sequences, such as promoter sequences, enhancer sequences, and other sequences that contribute to accurate or efficient transcription or translation, such as an internal ribosome binding site (IRES) or a polyadenylation (polyA) sequence, as well as additional transgenes. In embodiments, the polynucleotide sequence encoding the β-gal or an active variant thereof is operably linked to the promoter sequence. In some embodiments, the gene therapy vector comprises a polyA sequence but does not comprise an IRES sequence nor an additional transgene sequence.

In embodiments, the present disclosure provides a replication deficient AAV-derived vector comprising a polynucleotide sequence, e.g., an expression cassette, comprising the following in 5′ to 3′ order: a promoter sequence; a polynucleotide sequence encoding human β-gal or an active variant thereof; and a polyadenylation (polyA) sequence.

In embodiments, the promoter is a constitutive promoter, an inducible promoter, a tissue specific promoter (e.g., a brain-specific or neural tissue- or neural cell-specific promoter), or a promoter endogenous to the subject. Examples of constitutive promoters include, without limitation, the CMV early enhancer/chicken β actin (CAG) promoter, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter [Invitrogen]. In embodiments, the promoter is the CAG promoter, wherein the CAG promoter carries a CMV IE Enhancer, CB promoter, CBA Exon 1, CBA intron, rabbit beta-intron, and rabbit beta-globin exon 2.

Examples of inducible promoters regulated by exogenously supplied promoters include the zinc-inducible metallothionine (MT) promoter, the dexamethasone (Dex)-inducible mouse mammary tumor virus (MMTV) promoter, the ecdysone insect promoter, the tetracycline-repressible system , and the tetracycline-inducible system. Inducible promoters and inducible systems are available from a variety of commercial sources, including, without limitation, lnvitrogen, Clontech and Ariad. Many other systems have been described and can be readily selected by one of skill in the art.

IRES (Internal Ribosome Entry Site) are used in vectors containing an additional transgene. IRES are structural RNA elements that allow the translation machinery to be recruited within the mRNA, while the dominant pathway of translation initiation recruits ribosomes on the mRNA capped 5′ end. In embodiments, the vectors provided herein include neither an additional transgene nor an RIES.

The poly(A) signal is used by the cell for the 3′ addition of a polyA tail onto the mRNA. This tail is important for the nuclear export, translation, and stability of mRNA. In some embodiments, the polyA unit is a human growth hormone 1 poly A unit.

In embodiments of the vectors of the present disclosure, the promoter sequence is derived from CAG promoter sequence; and/or the polyA sequence is derived from a human growth hormone 1 polyA sequence.

In embodiments, the present disclosure provides a replication deficient AAV-derived vector comprising a polynucleotide sequence, e.g., an expression cassette, comprising the following in 5′ to 3′ order: a CAG promoter sequence; a polynucleotide sequence encoding human β-gal or an active variant thereof; and a polyadenylation (polyA) sequence derived from a human growth hormone 1 polyA sequence.

In embodiments, the present disclosure includes a composition comprising a gene therapy vector described herein and a pharmaceutically acceptable carrier, diluent or excipient. Such a composition may be referred to as a pharmaceutical composition. In one particular embodiment, the pharmaceutically acceptable carrier, diluent, or excipient is a phosphate buffered saline solution, which may be sterile and/or Good Manufacturing Practices (GMP) clinical grade.

In embodiments, the concentration of vector present in a composition of the present disclosure is about 1.0E+12 vg/mL to about 5.0E+13 vg/mL. For example, in embodiments, the concentration of vector present in the composition is about 1.0E+12 vg/mL, about 2.0E+12 vg/mL, about 3.0E+12 vg/mL, about 4.0E+12 vg/mL, about 5.0E+12 vg/mL, about 6.0E+12 vg/mL, about 7.0E+12 vg/mL, about 8.0E+12 vg/mL, about 9.0E+12 vg/mL, about 1.0E+13 vg/mL, about 2.0E+13 vg/mL, about 3.0E+13 vg/mL, about 4.0E+13 vg/mL, or about 5.0E+13 vg/mL.

In embodiments, the dose administered is from about 1.0E+12 vg/kg body weight to about 1.0E+13 vg/kg body weight. For example, in embodiments, the dose administered is about 1.0E+12 vg/kg, about 2.0E+12 vg/kg, about 3.0E+12 vg/kg, about 4.0E+12 vg/kg, about 5.0E+12 vg/kg, about 6.0E+12 vg/kg, about 7.0E+12 vg/kg, about 8.0E+12 vg/kg, about 9.0E+12 vg/kg, or about 1.0E+13 vg/kg. In embodiments, the dose administered is between about 3.0E+12 vg/kg and about 9.0E+12 vg/kg. In embodiments, the corresponding volume of CSF is estimated or calculated prior to administration. For example, in embodiments, the dose administered is about 3.2E+12 vg/kg body weight, corresponding to about 7.3E+11 vg/mL of CSF. In other embodiments, the dose administered is about 7.2E+12 vg/kg body weight, corresponding to about 1.8E+12 vg/mL of CSF.

In embodiments, a unit dosage form of the present disclosure comprises a vial containing about 500 μl to 20 mL of a composition of the present disclosure. In embodiments, a unit dosage form of the present disclosure comprises about 2 mL to about 12 mL. In embodiments, a unit dosage form comprises a vial containing about 500 about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL of the composition. In embodiments, the composition is administered at a flow rate of about 0.01 mL/min to about 5 mL/min. For example, in embodiments, the composition is administered at a flow rate of about 0.01 mL/min, about 0.05 mL/min, about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, about 1.0 mL/min, about 2.0 mL/min, about 3.0 mL/min, about 4.0 mL/min, or about 5.0 mL/min.

In embodiments, the gene therapy provided herein is administered via intracisternal injection, which is also referred to herein as injection into the cisterna magna, or ICM injection. ICM injection involves administration directly into the cerebrospinal fluid (CSF). It can be performed by direct injection, or via a catheter. In embodiments, ICM injection is performed with an infusion pump to control the rate of infusion.

In embodiments, the gene therapy is administered in a volume of about 0.1 mL/kg to about 2 mL/kg body weight. For example, the gene therapy is administered in a volume of about 0.1 mL/kg, about 0.2 mL/kg, about 0.3 mL/kg, about 0.4 mL/kg, about 0.5 mL/kg, about 0.6 mL/kg, about 0.7 mL/kg, about 0.8 mL/kg, about 0.9 mL/kg, about 1 mL/kg, or about 2 mL/kg. Thus, in embodiments, the present disclosure provides methods for treating GM1 gangliosidosis comprising administering a LYS-GM101 vector provided herein via ICM injection in a volume of about 0.5 mL/kg to about 1.0 ml/kg body weight, e.g., about 0.8 mL/kg body weight, e.g., between about 1 mL and about 20 mL, e.g., between about 2 mL and about 12 mL. In embodiments, a volume of CSF corresponding to about half of the volume of the ICM injection is removed prior to ICM injection.

### Polynucleotide and Polypeptide Sequences

In embodiments, the present disclosure includes polynucleotide sequences comprising or consisting of an expression cassette described herein, as well as plasmids and vectors comprising an expression cassette described herein. In addition, the disclosure includes cells comprising any of the polynucleotide sequences, vectors or plasmids of the present disclosure. One of skill in the art can readily produce polynucleotide sequences, vectors, and host cells of the present disclosure using standard molecular and cell biology techniques and knowledge in the art.

AAV cap sequences are known in the art. An exemplary AAVrh.10 cap polynucleotide sequence is provided as SEQ ID NO:59 in PCT Patent Application Publication No. WO2003/042397, with the sequence encoding VP1 at nucleotides 845-3061, VP2 at nucleotides 1256-3061, and VP3 at 1454-3061. An exemplary AAVrh.10 cap polypeptide sequence is provided as amino acid s 1-738 of SEQ ID NO:81 of PCT Patent Application Publication No. WO2003/042397, with the VP1 sequence at amino acids 1-738, VP2 at amino acids 138-738, and VP3 at amino acids 203-738.

In certain embodiments, a polynucleotide sequence comprising an expression cassette is present in a vector or plasmid, e.g., a cloning vector or expression vector, to facilitate replication or production of the polynucleotide sequence. Polynucleotide sequences of the present disclosure may be inserted into vectors through the utilization of compatible restriction sites at the borders of the ITR sequences or DNA linker sequences which contain restriction sites, as well as other methods known to those skilled in the art. Plasmids routinely employed in molecular biology may be used as a backbone, such as, e.g., pBR322 (New England Biolabs, Beverly, Mass.), pRep9 (Invitrogen, San Diego, Calif.), pB S (Stratagene, La Jolla, Calif.) for the insertion of an expression cassette.

Vectors or plasmids of the present disclosure may be present in a host cell, e.g., in order to produce the gene therapy vector or viral particles for clinical use. In particular embodiments, the present disclosure includes a cell comprising a vector or plasmid comprising an expression cassette of the present disclosure. In particular embodiments, the host cell is a 293 human embryonic kidney cell, such as, e.g., a 293T cell, a highly transfectable derivative of 293 cell that contains the SV40 T antigen. Examples of other vectors, host cells, and methods of producing viral vectors are described in Kotin R M, Hum Mol Genet, 2011 Apr. 15; 20(R1):R2-6. Epub 2011 Apr. 29).

In embodiments, the present disclosure includes gene therapy vectors or viral particles comprising any of the expression cassettes of the present disclosure, wherein said gene therapy vector or viral particle comprises a capsid, e.g., an AAVrh.10 capsid. In embodiments, the capsid comprises one or more AAVrh.10 capsid polypeptides.

In certain embodiments, polynucleotides, expression cassettes and vectors of the present disclosure may include an active variant of one or more active polynucleotide or polypeptide sequences, such as an active variant of a promoter sequence, an active variant of a polyA sequence, or an active variant of β-gal. Active variants include both biologically active variants and biologically active fragments of any of the sequences provided herein, which may be referred to as reference sequences. In particular embodiments, active variants of a reference polynucleotide or polypeptide sequence have at least 40%, 50%, 60%, 70%, generally at least 75%, 80%, 85%, usually about 90% to 95% or more, and typically about 97% or 98% or 99% or more sequence similarity or identity to the reference polynucleotide or polypeptide sequence, as determined by sequence alignment programs described elsewhere herein using default parameters. For example, in some embodiments, the present disclosure provides a polynucleotide having at least about 75%, 80%, 85%, 90%, 95%, 96%, 97%, 98%, or 99% sequence identity to any sequences provided herein, such as SEQ ID NOs: 1-6.

In embodiments, an active variant of a polynucleotide sequence encoding β-gal varies from a wild-type or naturally occurring gene or cDNA sequence due to degeneracy of the genetic code. Accordingly, while the polynucleotide sequence is varied from wild-type, the encoded β-gal retains the wild-type sequence. Thus, the present disclosure contemplates the use of any polynucleotide sequence that encodes the β-gal enzyme or active variants thereof

In embodiments, an active variant of a polynucleotide sequence that is active itself, e.g., a polyA sequence, may vary in sequence from its corresponding wild-type reference sequence, although it retains its native activity. An active variant of a reference polynucleotide sequence may differ from that sequence generally by as much 200, 100, 50 or 20 nucleotide residues, or suitably by as few as 1-15 nucleotide residues, as few as 1-10, such as 6-10, as few as 5, as few as 4, 3, 2, or even 1 nucleotide residue.

In embodiments, active variants of polypeptides are biologically active, that is, they continue to possess an enzymatic activity of a reference polypeptide. Such variants may result from, for example, genetic polymorphism and/or from human manipulation. An active variant of a reference polypeptide may differ from that polypeptide generally by as much 200, 100, 50 or 20 amino acid residues, or suitably by as few as 1-15 amino acid residues, as few as 1-10, such as 6-10, as few as 5, as few as 4, 3, 2, or even 1 amino acid residue. In some embodiments, a variant polypeptide differs from the reference sequences referred to herein by at least one but by less than 15, 10 or 5 amino acid residues. In other embodiments, it differs from the reference sequences by at least one residue but less than 20%, 15%, 10% or 5% of the residues.

A reference polypeptide may be altered in various ways including amino acid substitutions, deletions, truncations, and insertions to produce an active variant. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In embodiments, polypeptide variants contain conservative amino acid substitutions at various locations along their sequence, as compared to a reference polypeptide sequence. A “conservative amino acid substitution” is one in which the amino acid residue is replaced with an amino acid residue having a similar side chain. Families of amino acid residues having similar side chains have been defined in the art, which can be generally sub-classified as follows: acidic: the residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid; basic: the residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine; charged: the residues are charged at physiological pH and, therefore, include amino acids having acidic or basic side chains (i.e., glutamic acid, aspartic acid, arginine, lysine and histidine); hydrophobic: the residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan; and neutral/polar: the residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table 1.

TABLE 1
Amino acid sub-classification

| Sub-classes | Amino acids |
|---|---|
| Acidic | Aspartic acid, Glutamic acid |
| Basic | Noncyclic: Arginine, Lysine; |
| | Cyclic: Histidine |
| Charged | Aspartic acid, Glutamic acid, |
| | Arginine, Lysine, Histidine |
| Small | Glycine, Serine, Alanine, Threonine, Proline |
| Polar/neutral | Asparagine, Histidine, Glutamine, |
| | Cysteine, Serine, Threonine |
| Polar/large | Asparagine, Glutamine |
| Hydrophobic | Tyrosine, Valine, Isoleucine, |
| | Leucine, Methionine, |
| | Phenylalanine, Tryptophan |
| Aromatic | Tryptophan, Tyrosine, Phenylalanine, |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant polypeptide can readily be determined by assaying its enzymatic activity, as described herein. Conservative substitutions are shown in Table 2 under the heading of exemplary substitutions. Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary Amino Acid Substitutions

| Original Residue | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, | Leu |
| | Phe, Norleu | |
| Leu | Norleu, Ile, Val, Met, | Ile |
| | Ala, Phe | |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Thus, a predicted non-essential amino acid residue in a reference polypeptide is typically replaced with another amino acid residue from the same side chain family. A “non-essential” amino acid residue is a residue that can be altered from the wild-type sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of wild-type. An “essential” amino acid residue is a residue that, when altered from the wild-type sequence of a reference polypeptide, results in abolition of an activity of the parent molecule such that less than 20% of the wild-type activity is present. For example, such essential amino acid residues may include those that are conserved in the enzymatic sites of reference polypeptides from various sources.

In embodiments, the present disclosure also contemplates active variants of naturally-occurring reference polypeptide sequences, wherein the variants are distinguished from the naturally-occurring sequence by the addition, deletion, or substitution of one or more amino acid residues. In certain embodiments, an active variant of a polypeptide includes an amino acid sequence having at least about 50%, 55%, 60%, 65%, 70%, 75%, 80%, 85%, 90%, 91%, 92%, 93%, 94% 95%, 96%, 97%, 98% or more sequence identity or similarity to a corresponding sequence of a reference polypeptide described herein, and retains an enzymatic activity of that reference polypeptide.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

### Method for Producing Gene Therapy Vectors

Gene therapy vectors of the present disclosure may be produced by methods known in the art and previously described, e.g., in PCT Patent Application Publication No. WO03042397 and U.S. Pat. No. 6,632,670.

The AAV genome is a single-stranded deoxyribonucleic acid (ssDNA), either positive- or negative-sensed, which is about 4.7 kilobases long. The genome comprises ITRs at both ends of the DNA strand and two open reading frames (ORFs): rep and cap. Rep comprises four overlapping genes encoding Rep proteins required for the AAV life cycle, and cap comprises overlapping nucleotide sequences encoding capsid proteins: VP1, VP2 and VP3, which interact to form a capsid of an icosahedral symmetry.

The ITRs are believed to be required for both integration of the AAV DNA into the host cell genome and rescue from it, as well as for efficient encapsidation of the AAV DNA and generation of a fully-assembled AAV particles. With regard to gene therapy, ITRs seem to be the only sequences required in cis next to the therapeutic gene, and the structural (cap) and packaging (rep) genes can be delivered in trans. Accordingly, certain methods established for production of recombinant AAV (rAAV) vectors containing a therapeutic gene involve the use of two or three plasmids. In particular embodiments, the first plasmid comprises an expression cassette comprising a polynucleotide sequence encoding the therapeutic polypeptide, which contains flanking ITRs. In some embodiments, the second plasmid comprises rep and cap genes and flanking ITRs. In some embodiments, a third plasmid provides helper functions (e.g., from adenovirus serotype5). In order to generate recombinant AAV vector stocks, standard approaches provide the AAV rep and cap gene products on a plasmid that is used to cotransfect a suitable cell together with the AAV vector plasmid encoding the therapeutic polypeptide. In some embodiments, standard approaches provide the AAV rep and cap gene products on a plasmid that is used to cotransfect a suitable cell together with the AAV vector plasmid encoding the therapeutic polypeptide and together with the plasmid providing helper functions.

In embodiments, AAV rep and cap genes are provided on a replicating plasmid that contains the AAV ITR sequences. In embodiments, the rep proteins activate ITR as an origin of replication, leading to replication of the plasmid. The origin of replication may include, but is not limited to, the SV40 origin of replication, the Epstein-Barr (EBV) origin of replication, the ColE1 origin of replication, as well as others known to those skilled in the art. Where, for example, an origin of replication requires an activating protein, e.g., SV40 origin requiring T antigen, EBV origin requiring EBNA protein, the activating protein may be provided by stable transfection so as to create a cell line source, e.g., 293T cells), or by transient transfection with a plasmid containing the appropriate gene.

In other embodiments, AAV rep and cap genes may be provided on a non-replicating plasmid, which does not contain an origin of replication. Such non-replicating plasmid further insures that the replication apparatus of the cell is directed to replicating recombinant AAV genomes, in order to optimize production of virus. The levels of the AAV proteins encoding by such non-replicating plasmids may be modulated by use of particular promoters to drive the expression of these genes. Such promoters include, inter alia, AAV promoters, as well as promoters from exogenous sources, e.g., CMV, RSV, MMTV, E1A, EF1a, actin, cytokeratin 14, cytokeratin 18, PGK, as well as others known to those skilled in the art. Levels of rep and cap proteins produced by these helper plasmids may be individually regulated by the choice of a promoter for each gene that is optimally suited to the level of protein desired.

Standard recombinant DNA techniques may be employed to construct the helper plasmids used to produce viral vector of the present disclosure (see e.g., Current Protocols in Molecular Biology, Ausubel., F. et al., eds, Wiley and Sons, New York 1995), including the utilization of compatible restriction sites at the borders of the genes and AAV ITR sequences (where used) or DNA linker sequences which contain restriction sites, as well as other methods known to those skilled in the art.

In embodiments, gene therapy vectors of the present disclosure are produced by the transfection of two or three plasmids into a 293 or 293T human embryonic kidney cell line. In embodiments, DNA coding for the therapeutic gene is provided by one plasmid, and the capsid proteins (from AAVrh.10), replication genes (from AAV2) and helper functions (from adenovirus serotype5) are all provided in trans by a second plasmid. In embodiments, DNA coding for the therapeutic gene is provided by one plasmid, the capsid proteins (from AAVrh.10) and replication genes (from AAV2) are provided in trans by a second plasmid, and helper functions (from adenovirus serotype5) are provided by a third plasmid. In particular embodiments, the first plasmid comprises an expression cassette of the present disclosure, including the flanking ITRs.

Following cell culture, the gene therapy vector is released from cells by freeze thaw cycles, purified by an iodixanol step gradient followed by ion exchange chromatography on Hi-Trap QHP columns. The resulting gene therapy vector may be concentrated by spin column. The purified vector may be stored frozen (at or below −60° C.), e.g., in phosphate buffered saline.

Characterization of the final formulated vector may be achieved through SDS-PAGE and Western blot for capsid protein, real time PCR for transgene DNA, Western analysis, in vivo and in vitro general and specific adventitious viruses, and enzymatic assay for functional gene transfer.

### Methods of Treatment

The present disclosure provides methods of treating brain diseases and disorders, neurological diseases and disorders, and genetic diseases and disorders, including, but not limited to, lysosomal storage diseases. For example, the present disclosure provides methods of treating GM1 gangliosidosis comprising providing to a subject in need thereof a composition comprising a gene therapy vector designed to express β-gal when taken up by cells of the subject. In embodiments, the composition further comprises a pharmaceutically acceptable carrier, excipient or diluent, e.g., phosphate-buffered saline. In embodiments, a subject is a mammal, such as a human. In embodiments, the human is an adult, or the human is not an adult. In embodiments, the human is between 0 days and 18 years of age. In embodiments, the human is between 0 days and 6 months of age, or is between 6 months and 3 years of age, or is between 3 years and 6 years of age, or is between 6 years and 12 years of age, or is between 12 years and 18 years of age. In embodiments, a subject has been diagnosed with GM1 gangliosidosis, e.g., through genetic testing to identify a mutation in the subject's GLB1 gene or by measuring β-gal activity from a biological sample obtained from the subject. In embodiments, the methods provided herein restore at least about 1%, at least about 5%, at least about 10%, at least about 15%, at least about 20%, at least about 25%, at least about 30%, or more of normal β-gal activity throughout the brain of the subject. In certain embodiments, the methods provided herein restore at least about 20% of normal β-gal activity in the brain of the subject.

In certain embodiments, the composition comprising a gene therapy vector provided herein is administered to the subject's brain and/or spinal cord. In embodiments, the gene therapy vector provided herein is administered to the subject's CSF. For example, in some embodiments, the composition comprising the gene therapy vector is administered via intraventricular or intracisternal (ICM) injection. Injections may be accomplished in a single neurosurgical session. Injections may be performed by direct injection, or through an implanted catheter connected to an infusion pump. The infusion pump controls the rate of delivery.

In various embodiments in this disclosure, the term or unit genome copies (gc) is used interchangeably with the term or unit viral genomes (vg).

In certain embodiments, a total of about 1.0×10¹¹ g to about 1.0×10¹⁵ vg, about 5.0×10¹¹ vg to about 5.0×10¹⁴ vg, about 5.0×10^{12 vg} to about 1.0×10¹⁴ vg, about 1.0×10¹² vg to about 1.0×10¹⁴ vg, about 1.0×10¹³vg to about 5.0×10¹⁴ vg or about 5.0×10¹³vg to about 5.0×10¹⁴ vg of viral vector is administered to the subject.

In embodiments, the gene therapy vector LYS-GM101 is a solution for injection. In embodiments, the gene therapy vector is administered in a formulation comprising a PBS buffer. In embodiments, the PBS buffer is supplemented with 0.001% poloxamer (Kolliphor® P188). In some embodiments, the PBS buffer does not comprise any excipients or preservatives. In some embodiments, the composition of the PBS buffer comprises KCl, KH₂PO₄, NaCl, and/or Na₂HPO₄. In some embodiments, the composition of the PBS buffer comprises about 2.67 mM KCl, about 1.47 mM KH₂PO₄, about 137.9 mM NaCl, and about 8.06 mM Na₂HPO₄. In some embodiments, the pH of the formulation is about 6.8 to about 7.8, or about 7.2-7.4.

In embodiments, the present disclosure provides a method of treating GM1 gangliosidosis, said method comprising administering to a subject in need thereof (e.g., a human diagnosed with GM1 gangliosidosis), via ICM injection, a composition comprising a viral vector comprising an expression cassette comprising the following sequence in 5′ to 3′ order: a promoter sequence derived from a CAG promoter sequence, a polynucleotide sequence encoding human β-gal or an active variant thereof, and a human growth hormone 1 polyA sequence.

Accordingly, in embodiments, the present disclosure includes a method of treating a brain or neurological disease or disorder resulting from a mutated GLB1 gene in a subject in need thereof, comprising ICM administration to the subject of a gene therapy vector comprising an expression cassette comprising a polynucleotide sequence encoding the polypeptide encoded by the gene in its wild-type or non-mutated form, or an active variant thereof, wherein said polynucleotide sequence is operably linked to a promoter sequence, and wherein said ICM administration comprises administering about 1×10¹³ vg to about 5×10¹⁴ vg, or about 5.0×10¹³ vg to about 1.2×10¹⁴ vg, in a volume of about 0.5 mL/kg to about 1.5 mL/kg. For example, in a patient weighing about 5 kg (e.g., an infant), the gene therapy vector may be administered in a volume of about 2 mL; in a patient weighing about 15 kg, the gene therapy vector may be administered in a volume of about 6 mL. In embodiments, the polynucleotide sequence is operably linked to a CAG promoter. In embodiments, the ICM administration is performed using a delivery device, optionally comprising a catheter. In embodiments, the administration is via a catheter. In embodiments, the ICM administration is performed using an infusion pump.

In embodiments, the methods provided herein comprise administration of a gene therapy provided herein in combination with one or more immunosuppressants. In embodiments, the immunosuppressants are administered to the subject in need of the gene therapy provided herein prior to and/or concurrently with and/or subsequent to administration of the gene therapy vector. In embodiments, one or more of the immunosuppressants comprises a calcineurin inhibitor (e.g., tacrolimus), a macrolide (e.g. sirolimus or rapamicyn), and/or mycophenolate mofetil. In embodiments, one or more of the immunosuppressants comprises a steroid (e.g., prednisolone). In embodiments, one or more of the immunosuppressants is administered for at least 1, at least 2, at least 3, at least 6, or at least 12 months immediately following administration of the gene therapy vector. In embodiments, one or more of the immunosuppressants is administered for the remainder of the subject's life, or for as long as the subject is producing a detectable level of β-gal from the expression cassette.

All documents cited in this application are herein incorporated by reference in their entireties for all purposes

The present disclosure is further illustrated by reference to the following Examples. It should be noted that these Examples, like the embodiments described above, are illustrative and are not to be construed as restricting the scope of the disclosure in any way.

### EXAMPLES

### Example 1: LYS-GM101 Gene Therapy Vector

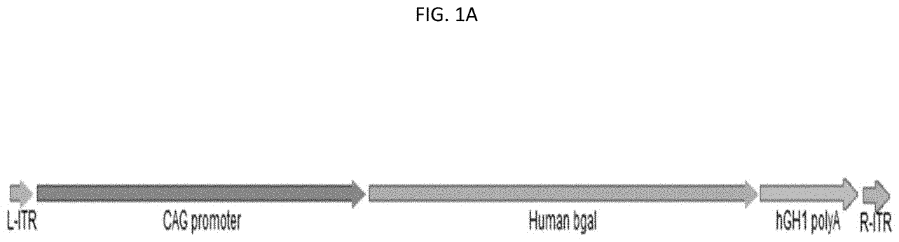

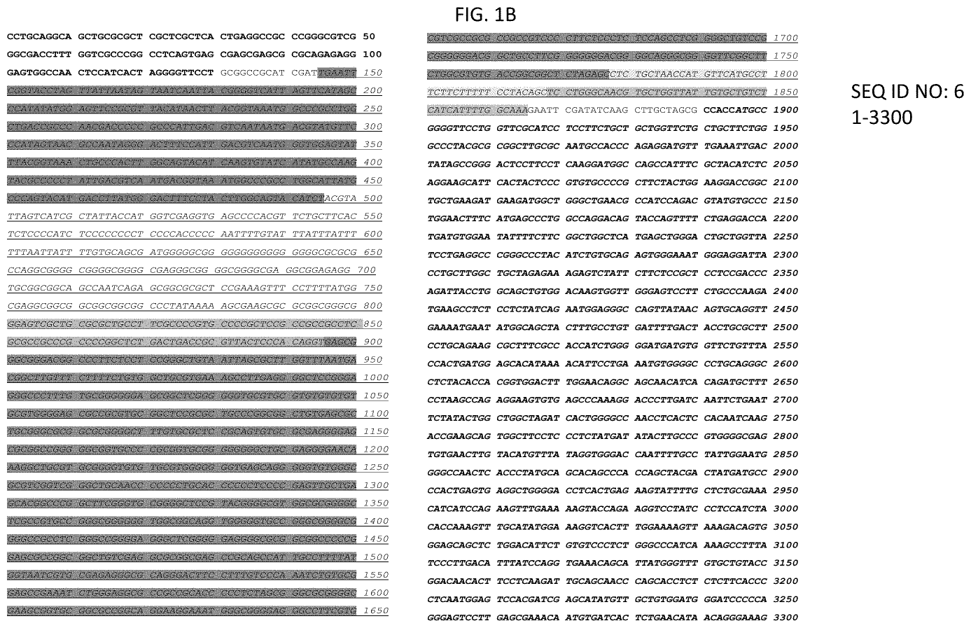

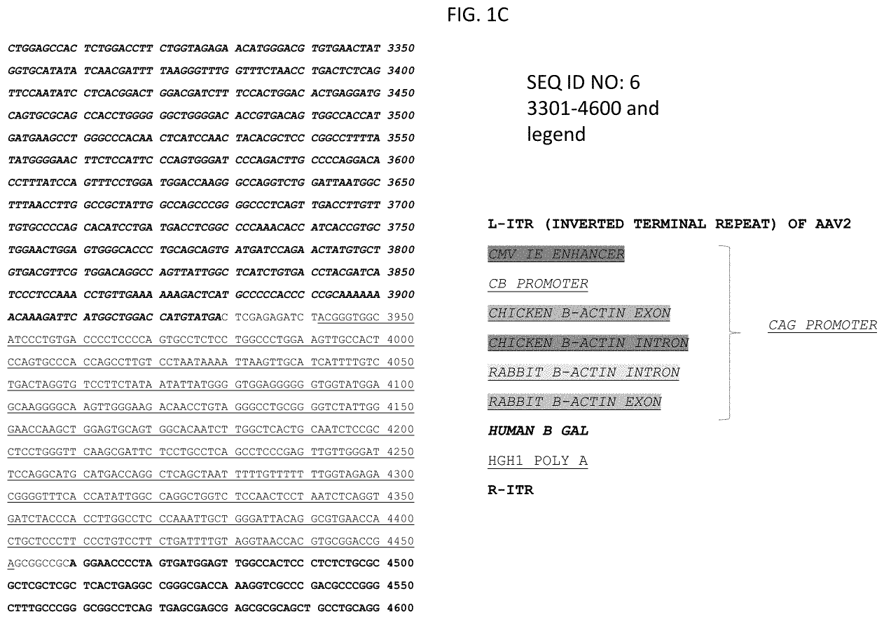

LYS-GM101 is a replication-defective recombinant AAVrh.10 vector that carries the human GLB1 gene driven by cytomegalovirus enhancer fused to a chicken β-actin promoter/rabbit β globin intron (CAG promoter), and the human growth hormone poly A sequence. The expression cassette including the promotor, GLB1 cDNA, and polyA sequence is flanked by AAV2 inverted terminal repeats. A schematic representation of the promoter, hGLB1 transgene, poly A sequence, and flanking sequences on the LYS-GM101 plasmid is provided as FIG. 1A. A table of the features and SEQ ID NOs for each feature of the plasmid is provided below in Table 3. The sequence of the plasmid is provided herein as SEQ ID NO: 6 (FIG. 1B and FIG. 1C).

TABLE 3
Table of GM-101 components

| | | SEQ ID |
|---|---|---|
| Feature | Description | NO |
| L-ITR | Left Inverted terminal repeat sequence | 4 |
| | from AAV serotype 2 | |
| Promoter | CAG promoter carrying a CMV IE | 2 |
| | Enhancer, CB promoter, CBA Exon 1, | |
| | CBA Intron, Rabbit beta-intron, | |
| | Rabbit beta-globin exon 2 | |
| Gene of | Human GLB1 | 1 |
| interest | | |
| Poly A | Human GH1 poly A | 3 |
| R-ITR | Right Inverted terminal repeat sequence | 5 |
| | from AAV serotype 2 | |

The expression cassette comprises, in order, a CMV early enhancer/chicken β actin (CAG) promoter, cDNA for the human GLB1 gene (hGLB1) encoding the lysosomal acid beta-galactosidase (β-gal) enzyme, and a human growth hormone 1 poly A unit (hGH1 polyA). A first AAV2 inverted repeat (ITR) containing 145 nucleotides and a second AAV2 ITR containing 145 nucleotides flank the expression cassette on either side. The two ITR termini are the only cis-acting elements required for genome replication and packaging. The hGH1 poly A unit is involved in mRNA stability and nuclear export towards mRNA translation.

LYS-GM101 DNA consists of 4.60 kb and the molecular weight is 1422.5 kDa. The β-gal sequence consists of 2.03 kb, and the molecular weight of the GLB1 DNA sequence is 627.5 kDa.

### Example 2: Dose Response Study of Intra-Thalamic or Cerebroventricular Injections of Murine LYS-GM101 in GM1 Gangliosidosis Mice

A study was conducted to establish a dose response for intra-thalamic and ICV routes independently. The study was a dose-response study for intra-thalamic (Thal) or intracerebroventricular (ICV) injection of a murine version of LYS-GM101 (AAVrh.10-mβgal) conducted in GM1 gangliosidosis mice.

The GM1 gangliosidosis knockout mouse (Hahn et al. 1997) is a well-established model of GM1 gangliosidosis disease. A large insertion in exon 6 of the GLB1 gene results in a truncated β-galactosidase protein and lack of β-gal activity. By 5 weeks of age, extensive lysosomal storage defects are seen in the brain and spinal cord, and pathology progresses over the next few months. Despite lysosomal dysfunction, the GM1 gangliosidosis mice show no clinical phenotype until about 5 months of age, when ataxia, tremor and abnormal gait become evident. The knockout mouse model replicates several clinical and biochemical features of infantile GM1 gangliosidosis, with low levels of β-gal activity and massive accumulation of GMlganglioside throughout the CNS (Baek et al. 2010). Thus, while lysosomal pathology indicates this model is the equivalent of human early infantile disease, neurological disease progression in mouse is slower than in humans.

GM1 gangliosidosis mice were injected bilaterally into the thalamus (2×2.2 μL) or unilaterally into the lateral ventricle (14.8 μL) with increasing doses of AAVrh.10-mβgal (Tha1: 3.5E+09, 1.0E+10, 3.5E+10, 1.0E+11 vg; ICV: 3.5E+10, 1.0E+11, 3.5E+11 vg) (Table 4). The choice of these sites of injections and doses were based on previous work in GM1 gangliosidosis mice using AAV1 coding for mβ-gal, which showed enzymatic and neurochemical correction in the CNS of treated animals (Baek et al. 2010; Broekman et al. 2007). PBS-injected GM1 gangliosidosis mice served as negative controls (same sites and volumes injected as vector injected groups). Four to six mice (both genders) were injected per group. Mice were injected at 6-8 weeks of age and euthanized at one-month post-injection, and tissues were collected for biochemical and histological analysis. Potential toxicity was also assessed by histopathology analysis of brain sections.

TABLE 4
Dose Response Study in GM1 Gangliosidosis Mice: Study Doses

| | Delivery route | |
|---|---|---|
| | Bilateral Thalamic (2.2 μL) | ICV (14.8 μL) |
| AAVrh10-mβgal | 3.5E+09 | NA |
| dose (vg) | 1.0E+10 | NA |
| | 3.5E+10 | 3.5E+10 |
| | 1.0E+11 | 1.5E+11 |
| | NA | 3.5E+11 |

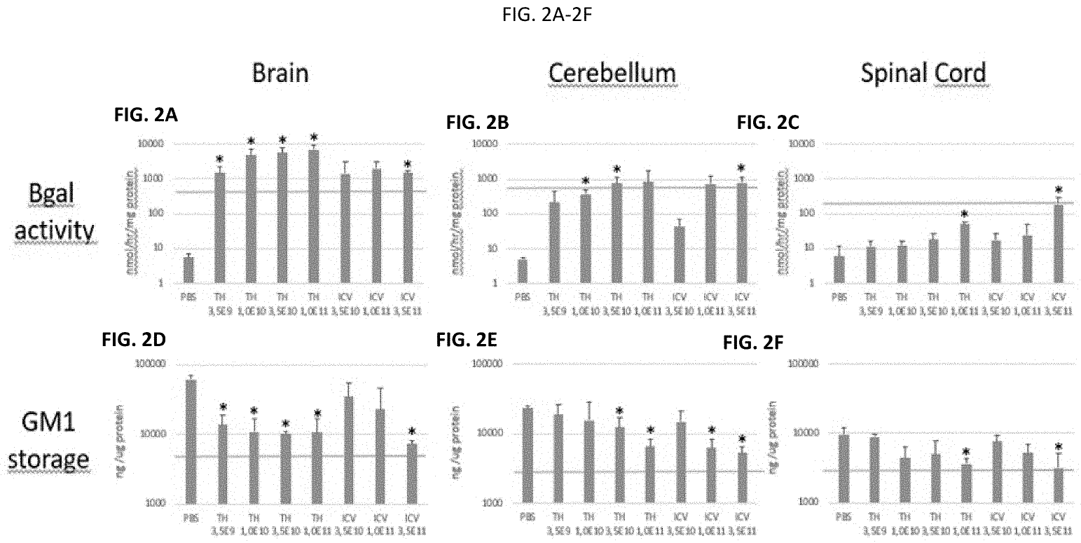

Quantitative assays were performed to measure β-gal enzyme activity and GM1 ganglioside content in the brain, cerebellum and spinal cord. Results presented in FIG. 2A-2F indicate that AAVrh.10-mβgal produced significant and dose-dependent increases in β-gal enzymatic activity and decrease of GM1 ganglioside content across all brain areas following thalamic injections, with a less clear dose response for the ICV injections. The lowest dose of 3.5E+09 vg used in intra-thalamic delivery led to a significant increase of β-gal activity, indicating that a minimum effective dose (MED) was not reached. ICV delivery (mid and high doses) resulted in comparable β-gal enzyme activity and GM1 ganglioside levels in the cerebellum, and higher effect in the spinal cord compared to intra-thalamic injection. An ICV dose of 3.5E+11 vg was needed to achieve a similar reduction in cerebral GM1 ganglioside content as that achieved by intra-thalamic injection at the lowest dose.

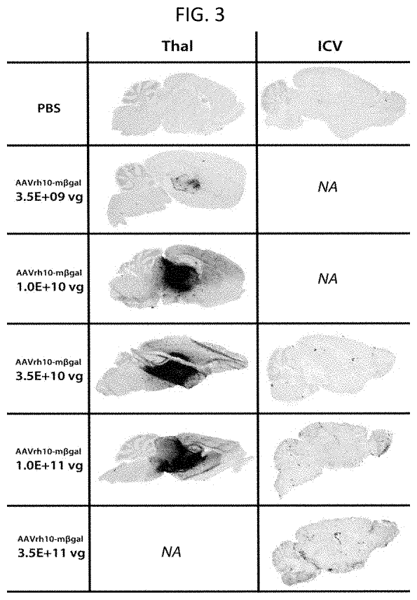

Histochemical staining with X-gal (FIG. 3) showed a dose dependent increase of β-gal enzyme activity in the brain of AAVrh.10-mβgal-injected animals. Intense staining and distribution radiating from the thalamic injection site were observed. After ICV injection, even at the highest dose, staining was much less intense but seemed more broadly distributed, reaching areas that were not stained after thalamic injection, such as the cerebellum. Direct intra-thalamic injection, but not ICV injection, resulted in dose-dependent toxicity at the two highest doses (3.5E+10 vg and 1.0E+11 vg) near the site of injection. More severe histopathologic changes were observed at the intra-thalamic dose of 1.0E+1 lvg needed to relieve storage defect in spinal cord. It should be noted that intra-thalamic injection of AAV vectors has been previously described to give rise to neuronal damage. On the other hand, no toxicity was observed following ICV injection even at the highest dose (3.5E+11 vg) that was associated with a positive pharmacological effect in all CNS compartments.

In summary, the study showed that ICV injection of AAVrh.10-mβgal, but not intra-thalamic injection, resulted in widespread (cerebrum, cerebellum and spinal cord) correction of storage defects at a dose that is free of observable adverse effects.

### Example 3: Feline LYS-GM101 in GM1 Gangliosidosis Cat Route Comparison Study

The effect of AAVrh.10-fβgal (feline analog of LYS-GM101) in restoring β-gal levels and reducing GM1 ganglioside in the CNS is provided herein in the following two studies using a well-characterized feline model of GM1 gangliosidosis (Martin et al. 2008)). This model resembles the juvenile form of the human disease. Onset of clinical neurological disease in affected cats occurs at approximately 3.5 months of age with a fine head or limb tremor. GM1 gangliosidosis mutant cats have progressive motor and ambulatory difficulties, with blindness and seizures in the terminal disease stage at 9-10 months of age.

The initial study in the feline model was conducted to explore three routes of administration: ICM, ICV and ITL. Based on the results of this first study, the second study (provided in Example 4) was conducted to evaluate the long-term efficacy of AAVrh.10fβgal delivered at high dose via the most promising CSF route, i.e. ICM, in GM1 gangliosidosis cats.

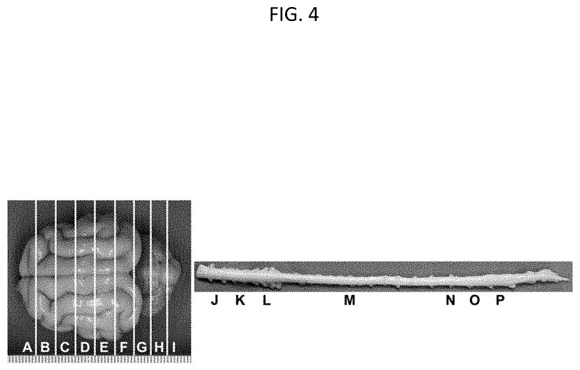

First, an efficacy and administration route comparison study of a feline version of LYS-GM101 was conducted in GM1 gangliosidosis cats. In this study, various routes of CSF delivery were evaluated for their potential to impact CNS distribution and β-gal enzyme levels. AAVrh.10-fβgal was delivered to GM1 gangliosidosis cats at a total dose of 1.0E+12 vg/kg body weight via one of three routes: ICM (n=4 both gender), ICV (n=4 both gender) or ITL (n=4 both gender). Cats were treated at 2-5 months of age and euthanized at one-month post-injection. Untreated GM1 gangliosidosis cats (n=4 both gender) and WT cats (n=4 both gender) were used as controls. For biochemical analysis, the brain and the spinal cord were collected and divided as shown in FIG. 4.

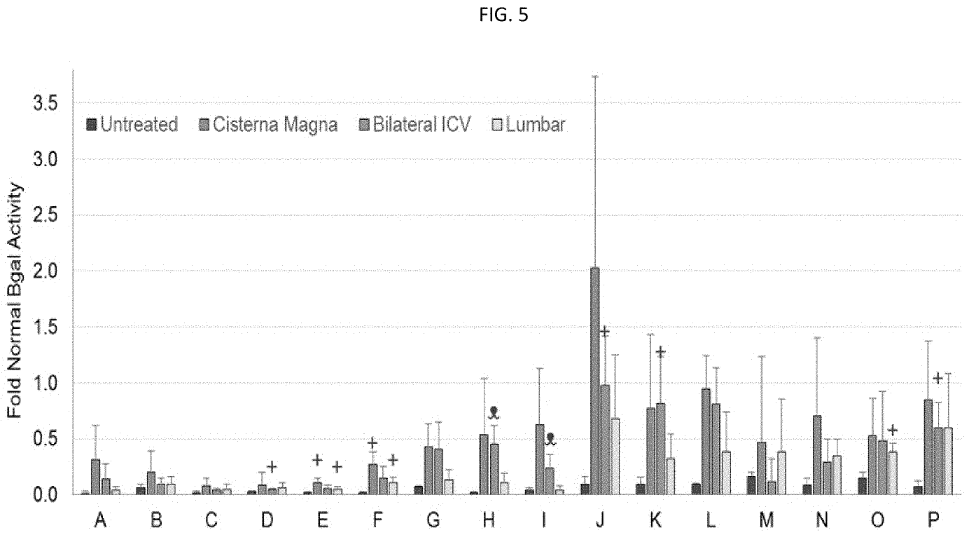

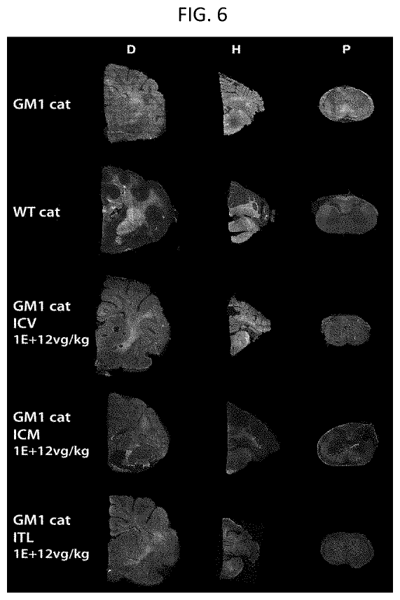

Quantitative assays were performed to measure β-gal enzyme activity in the cerebrum, cerebellum and spinal cord. β-gal enzyme activity is expressed as ‘fold normal’ levels, meaning that β-gal enzyme activity in each CNS block from treated animals was expressed relative to levels in the corresponding block from normal (WT) animals (n=3). Results are presented in FIG. 5 and indicate that bilateral ICV and ICM infusions of AAVrh.10-fβgal produced elevations in β-gal enzyme activity in cerebrum, cerebellum and spinal cord relative to untreated GM1 gangliosidosis cat tissues. While ITL delivery produced elevations in β-gal enzyme activity in spinal cord, this route was ineffective at delivering β-gal to the brain and cerebellum. In general, the highest β-gal enzyme activity in both the brain and spinal cord resulted from ICM infusion, ranging from 0.08-0.62-fold normal WT levels in the brain and 0.47-2.0-fold normal WT levels in the spinal cord.

β-gal activity was also measured in CSF, where mean activity increased after CM or ICV injection (range from 0.5-2.7-fold normal). The highest level of β-gal activity in peripheral organs was measured in the liver, where mean values were similar across injection routes and ranged from 0.72-1.1-fold normal. In addition, heart β-gal activity showed significant elevations after treatment by CM (0.45-fold normal) or ICV (0.32-fold normal) routes, with no elevation after ITL injection. The β-gal activity increase in peripheral organs indicates that vector can leak from the CSF to the blood and then transduce peripheral organs. This peripheral β-gal activity increase especially in liver and heart could have a beneficial impact on somatic symptoms that can be associated with GM1 gangliosidosis, such as cardiomyopathy and hepatosplenomegaly (Regier et al. 2016).

To evaluate lysosomal storage, filipin staining of the CNS was performed in a subset of treated GM1 gangliosidosis cats (FIG. 6). Visualized as punctate white or light gray dots, filipin staining is absent in the gray matter of the normal cat CNS, while prominent filipin staining is observed in the gray matter of the cerebrum, cerebellum, brainstem and spinal cord of untreated GM1 gangliosidosis cats. Filipin staining was diminished in the lumbar spinal cord of AAVrh.10-fβgal-treated GM1 gangliosidosis cats, demonstrating partial clearance of storage material in all treated cats, regardless of the route of injection. However, only the cats treated by ICM injection had effective clearance in the cerebellum and brainstem, with partial clearance in the cerebrum. In contrast, the cerebrum, cerebellum and brainstem were not effectively cleared of storage material in cats treated by the ICV or lumbar routes in this study.

In summary, this study showed that CSF administration of AAVrh.10 vectors in a large animal model can provide widespread CNS delivery of β-gal. Despite a limited increase of enzyme activity at 1.0E+12 vg/kg, especially in the brain, this study showed that ICV and ICM administration are preferable over lumbar delivery in elevating β-gal activity in the brain. The highest β-gal enzyme activity and associated clearance of storage in both the brain and spinal cord resulted from ICM infusion.

### Example 4: Long Term Efficacy of ICM Infusion of Feline LYS-GM101 in GM1 Gangliosidosis Cats

Based on the data provided by the study discussed above in Example 3, a long-term efficacy study of the feline version of LYS-GM101 delivered at high dose by ICM infusions was conducted in two juvenile male GM1 gangliosidosis cats.

AAVrh.10-fβgal was delivered to 2-3 months old GM1 gangliosidosis cats (n=2) via ultrasound guided, stereotaxic ICM infusion at a dose of 1.5E+13 vg/kg body weight. A 15-fold higher dose compared to the initial study was selected in order to increase the levels of β-gal activity in the CNS of treated GM1 gangliosidosis cats. Juvenile animals were used to allow treatment prior to first clinical sign. Untreated GM1 gangliosidosis cats (n=5) and WT cats (n=5) were used as controls. Cats were evaluated every 2 weeks for disease progression using a clinical rating scale (Table 5), up to the humane endpoint defined by the inability to stand on two consecutive days that is reached by untreated GM1 gangliosidosis cats at 8.0 (±0.6) months (Gray-Edwards, Regier, et al. 2017; McCurdy et al. 2014).

TABLE 5
Symptom onset in untreated GM1 gangliosidosis cats

| Score | Clinical status | Age (months) |
|---|---|---|
| 10 | Normal | <3.8 ± 0.3 |
| 9 | Fine tremors | 3.8 ± 0.3 |
| 8 | Hind limb weakness | 4.8 ± 0.5 |
| 7 | Wide stance | 5.4 ± 0.3 |
| 6 | Overt tremors | 5.4 ± 0.2 |
| 5 | Ataxia | 5.7 ± 0.3 |
| 4 | Limb spasticity | 0.6 ± 0.7 |
| 3 | Instability with occasional falling | 6.3 ± 0.5 |
| 2 | walk at least 4 steps | 7.1 ± 0.5 |
| 1 | Can stand but not walk | 7.3 ± 0.4 |
| 0 | Cannot stand | 8.0 ± 6.0 |

(Gray-Edwards, Regier, et al. 2017; McCurdy et al. 2014)

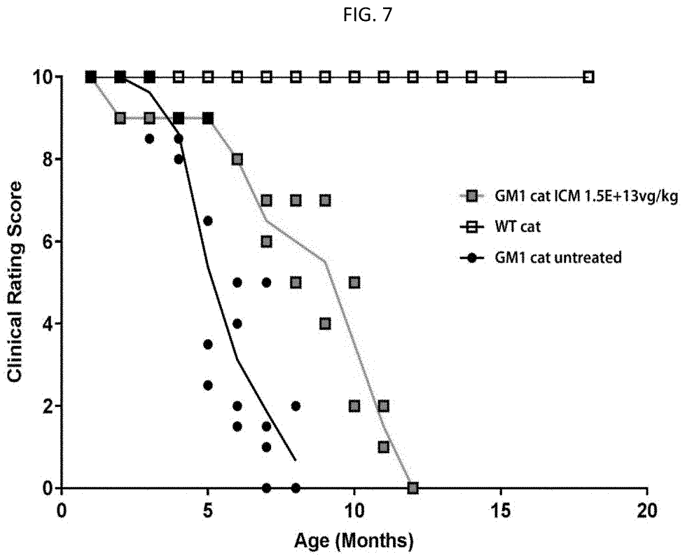

AAVrh.10-fβgal ICM injected cats survived significantly longer than untreated GM1 gangliosidosis cats with a mean lifespan of 11.3±0.7 months compared to 8.0±0.6 months for untreated GM1 gangliosidosis cats (p=0.0405, log rank Mantel-Cox test). Clinical rating scores are presented in FIG. 7 and show that clinical disease progression was delayed but not arrested by ICM injection of AAVrh.10-fβgal. All animals became blind as their disease progressed, though blindness is not incorporated in the rating scale.

Magnetic resonance spectroscopy (MRS) measurements were performed in treated cats at 8.8 months and at humane endpoint. In previous studies in GM1 gangliosidosis cats, the most informative and consistent predictor of CNS disease progression has been glycerophosphocholine+phosphocholine (GPC+PC) combined level, which increases as myelin integrity is compromised (Gray-Edwards, Regier, et al. 2017). GPC+PC in treated cats was equivalent to, or greater than, levels in untreated GM1 gangliosidosis cats at humane endpoint in every voxel except cerebellum. In cerebellum, mean levels of GPC+PC decreased moderately after treatment at both 8.8 months and humane endpoint.

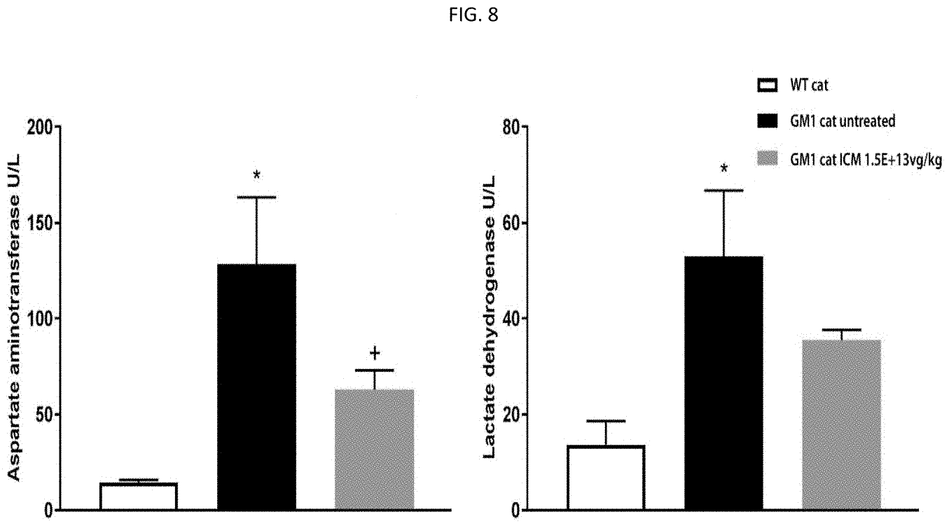

In addition to brain MRS to track the effect of gene therapy, markers of disease progression such as aspartate aminotransferase (AST) and lactate dehydrogenase (LDH) were measured in CSF. Though typically used to evaluate liver or muscle disease by measuring their levels in peripheral blood, AST and LDH also have been shown in previous work to correlate with neurodegeneration when measured in CSF of GM1 gangliosidosis cats (Gray-Edwards, Jiang, et al. 2017). As shown in FIG. 8, AST and LDH in CSF decreased to ˜50% of untreated levels for treated cats, though levels still remained above normal.

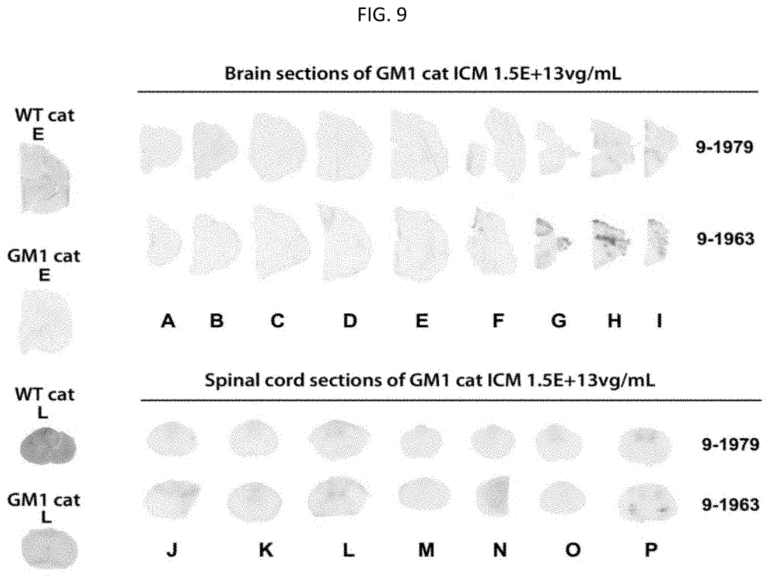

β-gal activity and biodistribution were evaluated by Xgal staining of 16 sections from the brain and spinal cord (FIG. 9). β-gal activity was broadly apparent in the cerebellum and spinal cord of treated GM1 gangliosidosis cats. However, little activity was detected in the cerebrum. The small amount of β-gal activity in the cerebrum was not detected in deep brain structures but was limited to areas directly exposed to CSF, such as sulci and periventricular regions.

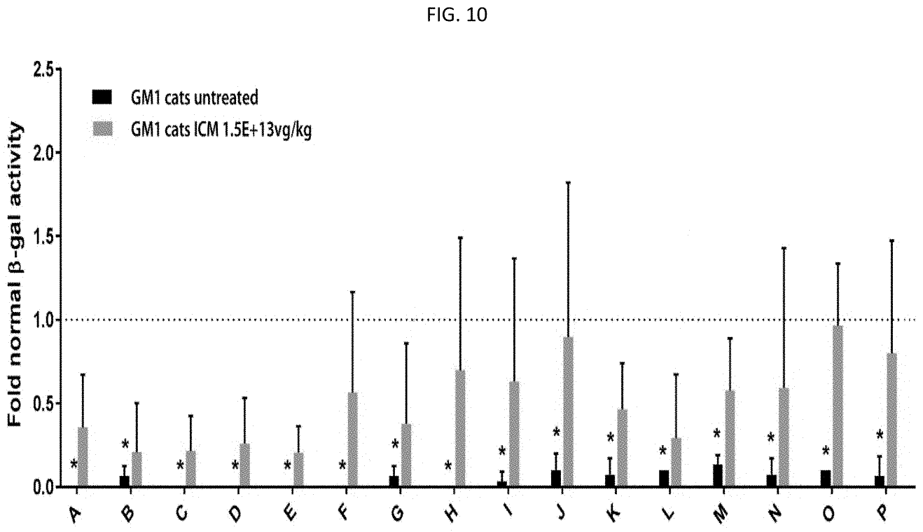

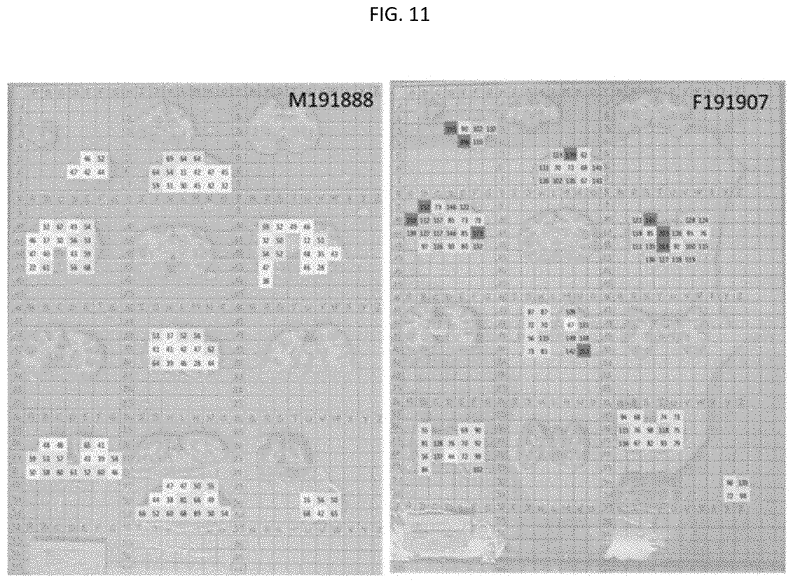

Quantitative assays confirmed the findings from Xgal staining, with low levels of β-gal activity in the cerebrum and higher levels in the cerebellum and spinal cord (FIG. 10). Levels ranged from 0.2-0.6-fold normal in the cerebrum, 0.4-0.7-fold normal in the cerebellum and 0.3-1.0-fold normal in the spinal cord.

Despite the 15-fold higher dose tested in this study compared to the earlier study, similar levels of β-gal activity were found in the brain (see FIG. 5) and the enzyme was virtually absent from deep brain structures. The cause of this unexpected result has not been determined, but it cannot be excluded that it was due to an error in viral titration and/or missed injections. The limited increase in β-gal activity observed in this study, especially in deep brain structures, could explain the incomplete correction of clinical phenotype in the treated cats.

In summary, this study showed that despite low levels of β-gal increase in the brain, ICM injection of the feline version of LYS-GM101 leads to clinical improvements in GM1 gangliosidosis cats that were associated with a decrease of neurodegeneration biomarkers in the CSF and MRS markers of neurodegeneration in the cerebellum.

### Example 5: β-gal Activity in the CNS of Juvenile Non-Human Primates (NHP) Following Single ICM Administration of LYS-GM101

β-gal enzyme activity in the CNS was evaluated in a GLP toxicology and biodistribution study conducted in juvenile NHP. The aim of the study was to determine toxicity and biodistribution of LYS-GM101 administered once into the cisterna magna of Cynomolgus monkeys. The study was conducted according to the design described in Table 6.

TABLE 6
NHP study design

| | Control | | Low dose | | High dose | | |
|---|---|---|---|---|---|---|---|
| | (group 1) | | (group 2) | | (group 3) | | |
| Treatment | M | F | M | F | M | F | Total |
| Sacrifice at Week 12 | 2 | 2 | 3 | 3 | 3 | 3 | 16 |
| (D78/D79) | | | | | | | |
| Sacrifice at Month 6 | 1 | 1 | 0 | 0 | 2 | 2 | 6 |
| (D181/D182) | | | | | | | |
| Total | 3 | 3 | 3 | 3 | 5 | 5 | 22 |

M: Male, F: Female (25-33 months of age).

LYS-GM101 or its vehicle were administered in one single session on D1 in the cisterna magna space by infusion of 4.5 mL at a flow rate of 0.5 mL/min at the following concentrations: Low dose—3.0E+12 vg/mL i.e. 1.4E+13 vg/animal; High dose—1.2E+13 vg/mL i.e. 5.4E+13 vg/animal

Based on studies in GM1 gangliosidosis mice and cat models described herein, relatively high doses of LYS-GM101 appear to be required for treatment efficiency. The maximum feasible dose of LYS-GM101 was therefore tested in NHP, based on the maximum feasible vector concentration of drug product batches and the maximum volume that can be safely injected into the NHP cisterna magna, 1.2E+13 vg/mL and 4.5 mL, respectively. Therefore, the maximum feasible dose (i.e., 5.4E+13 vg) and a 4-fold lower dose (i.e., 1.4E+13 vg) were tested to allow dose response observations.

LYS-GM101 or its vehicle was administered in one single session on D1 using a 20-gauge spinal needle manually inserted by palpation into the cisterna magna space of anaesthetized animals. Correct positioning was confirmed by the flow of CSF from the needle. The location of the needle was secured using a stereotaxic frame. The needle was connected to an infusion pump through an extension set of 1 m to allow infusion of 4.5 mL of test item or vehicle at a flow rate of 0.5 mL/min. At the end of the injection, the needle was left in place for 5 minutes to prevent reflow. The needle was then removed, and pressure was applied for about 30 seconds to the injection site.

On the day of necropsy (Week 12 (D78/D79)) and Month 6 (D181/D182) and after an overnight fast prior to sacrifice, animals were premedicated with ketamine HCl and euthanized by subtotal exsanguination following sodium pentobarbital anesthesia by the intravenous route.

Brain (perfused with cold sterile saline) was cut into 4 mm thick slices using a brain slicer. Odd slabs were fixed in buffered formalin for histopathology examination. Even slabs were divided into 10×10 mm sections and photographed (with scale) to document location of each section (FIG. 11). Each section was divided in half; one half was submitted for DNA quantification (Week 12 cohort only) and the other half for β-gal enzyme activity (both Week 12 and Month 6 cohorts). β-gal enzyme activity results from the Week 12 cohort are presented herein.

β-gal enzyme activity in CNS samples (99 to 123 brain samples and 3 spinal cord samples per animal) was quantified using a fluorometric enzymatic assay and results were expressed as nmol of product (4-MU) per hour and per mg of protein. The level of β-gal enzyme activity observed in vehicle treated animals (Group 1) corresponds to the endogenous activity of the enzyme in NHP and was considered as background level in Groups 2 and 3. In Group 1, the mean enzymatic activity was 52 nmol/h/mg of protein, with no significant difference between genders (mean of 53 nmol/h/mg for males and 51 nmol/h/mg for females).

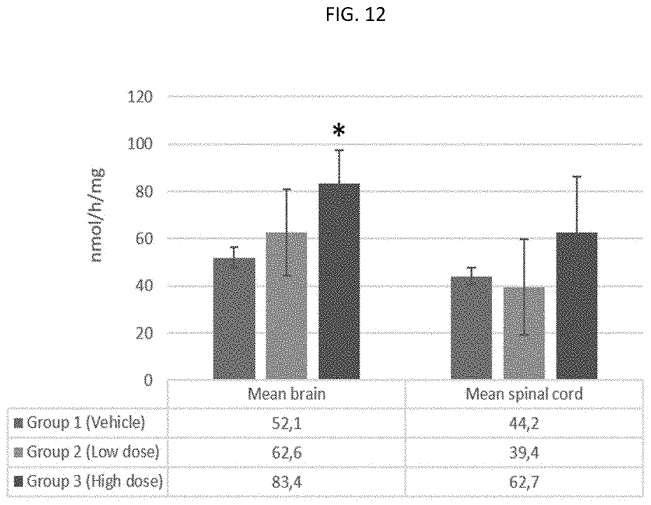

The measured activity of β-gal in brain samples showed heterogeneous values between brain sections and even between samples from a similar brain section as illustrated in FIG. 10. However, a global increase of enzyme activity was observed in the brain of both LYS-GM101-treated groups compared to the control group (20% and 60% increase for Group 2 and Group 3 respectively). This difference was statistically significant between Group 1 and Group 3, with mean values of 52.1 and 83.4 nmol/h/mg, respectively (p=0.002) (FIG. 12). Global β-gal activity increase observed in the brain was associated with an increase of the proportion of analyzed samples that showed ≥20% increase of β-gal activity over background levels, reflecting that β-gal activity increased throughout the brain rather than being limited to some only a few brain areas. In spinal cord sections, a 42% increase in β-gal activity was observed in Group 3 animals, relative to the mean values of Group 1, which did not however reach statistical significance (FIG. 12).

A mean of 68% (+/−16%) of analyzed brain samples from Group 3 animals showed ≥20% increase of β-gal activity over background levels. It should be noted that this level of increase of β-gal activity, if translated to infantile or juvenile GM1 gangliosidosis patients, would be expected to lead to a therapeutic effect. Indeed, disease severity in GM1 gangliosidosis correlates with residual enzyme activity, with infantile and juvenile patients expressing <1% and <10% of normal levels, respectively (Regier and Tifft 2013), and asymptomatic heterozygote subjects have a mean of 36-38% of normal β-gal activity in fibroblasts/leukocytes with a lower limit found at 16-19% (Sopelsa et al. 2000).

### Example 6. Summary of Non-Clinical Studies

Taken together, the results of the non-clinical studies established the qualitative principle that elevation of β-gal activity in the CNS via ICM administration of LYS-GM1010 leads to beneficial therapeutic effects in GM1 gangliosidosis.

Rather than extrapolating preclinical vector doses to human vector doses, dose selection for the clinical study provided below in Example 7 was based on a target engagement analysis. To select a dose of LYS-GM101 with expected clinical benefit, based on the information obtained in the preclinical studies provided herein, the inventors reasoned that this dose should lead to restoration of about 20% of normal β-gal activity in the central nervous system of patients with GM1 gangliosidosis patients. This is based on the following rationale. First, in GM1-gangliosidosis, there is a good correlation between residual enzyme activity and age of onset and severity of disease ((Regier and Tifft 2013) and Table 7).

TABLE 7
Correlation between residual enzyme activity and age of onset
and severity of disease (Regier and Tifft 2013)

| | GM1 Gangliosidosis | | | |
|---|---|---|---|---|
| | Type I | Type II | | Type III |
| | Early Infantile | Late Infantile | Juvenile | Chronic/Adult |
| β-galactosidase | Negligible | ~01%-5% | ~3%-10% | ~5%-10% |
| enzyme activity | | | | |

No disease is observed in carriers with greater than 10% residual enzyme activity. Consistently, asymptomatic heterozygote subjects have a mean of 36-38% of normal β-gal activity with a lower limit of 16-19% (Sopelsa et al. 2000). Second, Sandhoff and colleagues (Conzelmann and Sandhoff 1983); (Leinekugel et al. 1992); (Sandhoff and Harzer 2013), using an enzyme kinetic model of lysosomal substrate turnover, demonstrated that for most lysosomal enzymes significant decreases of enzyme activity can be tolerated without a significant effect on substrate turnover. It is only when enzyme activity decreases below a critical threshold that substrate will accumulate and lead to lysosomal storage pathology. For many lysosomal enzymes, this critical threshold occurs at 5-10% of normal average. In the case of GM2 gangliosidosis, substrate degradation rate in cells with varying degrees of residual enzyme activity was shown to increase steeply with residual activity, to reach normal levels at a residual activity of 10-15% of normal. All cells with an activity above this critical threshold had a normal turnover (Leinekugel et al. 1992). Similar observations were reported for metachromatic leukodystrophy, Gaucher, Sandhoff, and ASM-deficient Niemann-Pick disease (Sandhoff and Harzer 2013). Importantly, the correlation between residual enzyme activity and disease severity in GM1 gangliosidosis is very similar to that seen in GM2 gangliosidosis, such that the fact that healthy carriers of GLB-1 mutations can have residual activities as low as 16% is compatible with the enzyme kinetic model described by Sandhoff and colleagues.

In addition, some preclinical studies in GM1 gangliosidosis animal models demonstrate a relationship between enzyme activity following delivery of β-gal-expressing vectors (which reflects target engagement) and disease phenotype that confirms the notion of the ˜20% threshold. Thus, 10% to 20% of normal β-gal activity in the cerebrum of GM1 gangliosidosis mice treated with IV injection of AAV9-mβgal is sufficient to achieve significant biochemical impact with phenotypic amelioration and extension in lifespan (Weismann et al. 2015). Furthermore, using an ICV-administered AAV vector encoding human β-gal, restoration of a level of β-gal activity in the brain of GM1 gangliosidosis mice 2-3-fold lower than that of heterozygotes (which have about 50% of normal enzyme activity) had significant beneficial effects on neurological scores, lysosomal pathology and survival. In the cat study described above, significant clinical improvements were observed with brain β-gal activity levels lower than 50% on average. No conclusion as to the minimally effective level of target engagement could be drawn from mouse studies, since the lowest dose of AAVrh.10-mβgal used gave rise to brain β-gal activity higher than in wild-type animals.

Taken together, these results indicate that about 20% of normal enzyme activity is sufficient not only to prevent the development of disease in heterozygote human carriers of GLB-1 mutations (as discussed above), but also to correct or revert disease manifestations in the CNS of homozygote diseased animals, and presumably human patients. Even supplying just a few percent of normal activity to a patient with type I GM1 gangliosidosis could be beneficial, since this most severe form of the disease, with a life expectancy of 2-3 years, is associated with less than 1% residual activity, while the relatively milder juvenile and adult forms of disease are associated with 3 to 10% of residual activity (Regier and Tifft 2013).

Overall, the preclinical studies demonstrated that LYS-GM101 will provide clinical benefit. Doses of LYS-GM101 equivalent to the intended clinical doses are able to restore greater than 20% of normal β-gal activity in the brain and spinal cord of cynomolgus monkeys, whose CNS anatomy is similar to that of children. Since restoration of β-gal activity to levels 15-20% of normal is expected to halt substrate accumulation in cells of patients with GM1 gangliosidosis, the intended clinical doses of LYS-GM101 are expected to provide significant clinical benefit, including slowing of disease progression and possibly extending survival. Importantly, even restoration of a few percent of β-gal activity in cells of patients with GM1 gangliosidosis has the potential to convert the course of type I GM1 gangliosidosis to that of the milder juvenile or adult form of disease.

### Example 7. Human Clinical Study of LYS-GM101 Gene Therapy in Patients with GM1 Gangliosidosis

An exemplary open-label, adaptive-design study of intracisternal (ICM) administration of adeno-associated viral vector serotype rh.10 carrying the human β-galactosidase cDNA for the treatment of GM1 gangliosidosis is provided herein. The study is conducted in two stages: a safety and preliminary efficacy stage, and a confirmatory stage. The primary objective of the first stage is to assess the safety and tolerability of intracisternal administration of LYS-GM101 in early and late infantile GM1 gangliosidosis patients. The secondary objective of the first stage is to collect preliminary efficacy data and to select the primary efficacy endpoints and timepoints of primary interest for the second stage. Primary endpoint selection will be based on natural history data and preliminary efficacy data collected in infantile GM1 gangliosidosis patients during the first stage. The primary objective of the confirmatory stage is to demonstrate efficacy of intracisternal administration of LYS-GM101 in infantile GM1 gangliosidosis patients. The secondary objective of the confirmatory stage is to assess the safety and tolerability of LYS-GM101 in infantile GM1 gangliosidosis patients.

The first stage will enroll patients with early and late infantile GM1 gangliosidosis. An initial cohort of patients (including early and late infantile) will receive a potentially effective dose based on preclinical data with 2- to 5-fold safety margin relative to the highest dose (in vg/mL of CSF) tested in the GLP toxicology study. Enrollment of patients (including early and late infantile) in the 2^{nd} cohort will be initiated following review of one-month safety data post-administration per subtype within cohort 1 by an independent Data Safety Monitoring Board (DSMB). For each GM1 gangliosidosis subtype, additional patients will be enrolled in the event one patient shows toxicity.

After review by the DSMB of one-month safety and biomarker data on the first patients enrolled in cohort 2, the enrollment in cohort 2 will resume, marking the initiation of Stage 2 (confirmatory phase of the study).

Multiple safety and efficacy variables will be measured at 6 months to assess response to treatment. Endpoints, outcome measures, duration of follow-up, and timepoints of primary interest for each GM1 gangliosidosis subtype in the confirmatory phase of the study will be selected after interim analysis of the 6-month data in the 8 first patients enrolled in the study. All patients enrolled in Stage 1 will remain in the study for at least 2-years follow-up and will be included in the final analysis.

Considering the different patterns of progression between the early infantile and late infantile forms, different primary endpoints and timepoints for each group of patients may be selected for Stage 2. Based on the rapidity of decline described in natural history data, it is anticipated that timepoints of primary interest will be at one and two years for early infantile and late infantile, respectively. All patients will be followed for at least 2 years following LYS-GM101 administration.

Different GM1 gangliosidosis types will be analyzed separately. An interim analysis at one-year post administration is planned. Data will be compared to published historical natural history data in early infantile (Utz et al. 2017) and late infantile (Regier et al. 2015) GM1 gangliosidosis patients, as well as data from ongoing natural history studies (NCT 00668187, NCT03333200, NCT00029965) and registries.

After completion of the study, all patients will be asked to rollover into a long-term follow-up study of at least 3 years.

Inclusion criteria include:
1. β-gal gene mutations and/or documented deficiency of β-gal enzyme by laboratory testing.
2. Study population
   Children with early infantile GM1 gangliosidosis less than 12 months of age with ability to swallow (presence of feeding tube is permitted)
   Children with late infantile GM1 gangliosidosis less than 3 years of age with ability to sit with only arm support or with props
3. Signed written informed consent before any study related procedure is performed
4. Patient medical status sufficiently stable and ability of parents/legal guardian, in the opinion of the Investigator to adhere to the study visit schedule and other protocol requirements.

Exclusion criteria include:
1. Uncontrolled seizure disorder. Patients who are stable on anticonvulsive medications may be included
2. More than 40% brain atrophy as measured by MRI total brain volume at screening
3. Current participation in a clinical trial of another investigational medicinal product
4. Past participation in gene therapy trials
5. History of hematopoietic stem cell transplantation
6. Any condition that would contraindicate treatment with immunosuppressant therapy
7. Presence of concomitant medical condition or anatomical abnormality precluding lumbar puncture or intracisternal injection
8. Presence of any permanent items (e.g., metal braces) precluding undergoing MRI
9. History of non-GM1 gangliosidosis medical condition that would confound scientific rigor or interpretation of results
10. Rare and unrelated serious comorbidities, e.g., Down syndrome, intraventricular hemorrhage in the new-born period, or extreme low birth weight (<1500 grams)
11. Any vaccination 1 month prior to the planned immunosuppression treatment
12. Serology consistent with HIV exposure or consistent with active hepatitis B or C infection
13. Grade 2 or higher lab abnormalities for LFT, bilirubin, creatinine, hemoglobin, WBC count, platelet count, PT, and a PTT, according to CTCAE v5.0.

The investigational drug is LYS-GM101. LYS-GM101 is an adeno-associated viral vector serotype rh.10 (AAVrh.10) carrying the human GLB1 gene, formulated as a solution for injection. The volume of intra-cisterna magna injection is expected to range from 4 to 12 mL (0.8 mL per Kg of body weight).

Each patient will receive a single dose of LYS-GM101 via injection into the cisterna magna under imaging guidance. A volume of CSF corresponding to half of the drug volume to be injected will be removed before the infusion. In cohort 1, patient dose is 3.2E+12 vg/Kg, corresponding to 7.3E+11 vg/mL of CSF, and drug material for cohort 1 is at a concentration of 4.0E+12 vg/mL. The volume of injection will be 0.8 mL/kg and range from 4 mL (for a 3-month old child of 5 kg) to 12 mL (for a 36-month old child of 15 Kg). In cohort 2, patient dose is 8.0E+12 vg/Kg, corresponding to 1.8E+12 vg/mL of CSF, and drug material for cohort 2 is at concentration of 1.0E+13 vg/mL. The volume of injection will be 0.8 mL/kg and range from 4 mL (for a 3-month old child of 5 kg) to 12 mL (for a 36-month old child of 15 Kg).

After one-month post administration of the 4 first patients in cohort 1, data will be reviewed by the DSMB. In the absence of unexpected safety signal and in presence of positive biomarker readouts, all additional patients enrolled in the study will be treated. Patient dose will be calculated based on body weight up to 36 months of age (15 Kg).

All patients will receive short-term corticosteroids (prednisolone 1 mg/Kg/day) for 10 days with initiation 1 day before LYS-GM101 administration to prevent primarily immune reaction against the vector DNA. In addition, to prevent long-term immune reaction against the β-gal transgene, all patients will receive: Mycophenolate mofetil (oral solution) started 7 days before surgery and for 2 months post-administration (8 weeks); and Tacrolimus (granules for oral suspension or capsules) started 7 days before surgery and for at least 6 months post administration. Maintenance of long-term immunosuppression beyond 6 months will depend on the patients' β-gal enzyme level at baseline. As patients with null enzyme level potentially make no protein, the immunosuppressant (tacrolimus) will be continued, at very low doses to prevent immune reaction against the transgene, whereas patients with non-null residual enzyme level will be progressively discontinued approximatively 6 months post-administration. The tapering phase will be monitored with regular measurements of humoral and cellular immune responses to ensure safe discontinuation of tacrolimus.

The primary objective of Stage 1 is to assess the safety/tolerability of 2 doses of LYS-GM101 drug product. Safety and tolerability will be monitored by means of scheduled complete physical examinations (including height and weight), neurological exam, vital signs (including body temperature, pulse and blood pressure (BP) measurements), imaging (MM, X-ray, heart and abdominal ultrasounds), functional assessments (ECG, EEG with ERP, visual and hearing assessments), laboratory determinations (hematology, blood chemistry and coagulation), and collection of adverse events throughout the study. Safety evaluation will also include assessments of immunogenicity: anti-AAVrh.10 antibodies, anti-β-gal antibodies, and assessment of cellular immunity, particularly in case of immunosuppression discontinuation.

The secondary objective of Stage 1 is to collect and analyze a series of efficacy variables using standardized assessment tools for determination of appropriate efficacy endpoints for the confirmatory phase of the study. The primary and secondary efficacy endpoints for early and late infantile GM1 gangliosidosis patients will be confirmed when the first 8 patients have reached 6-month follow-up (interim analysis). They will be selected among the efficacy variables collected during Stage 1 based on the interim analysis at 6 months and supported by the natural history studies and registry data. It is expected that selected endpoints for the confirmatory phase will differ based on GM1 gangliosidosis clinical type.

### REFERENCES

Baek, R. C., M. L. Broekman, S. G. Leroy, L. A. Tierney, M. A. Sandberg, A. d'Azzo, T. N. Seyfried, and M. Sena-Esteves. 2010. ‘AAV-mediated gene delivery in adult GM1-gangliosidosis mice corrects lysosomal storage in CNS and improves survival’, PLoS One, 5: e13468.
Bartus, R. T. 2015. ‘Gene therapy for Parkinson's disease: a decade of progress supported by posthumous contributions from volunteer subjects’, Neural Regen Res, 10: 1586-8.
Broekman, M. L., R. C. Baek, L. A. Comer, J. L. Fernandez, T. N. Seyfried, and M. Sena-Esteves. 2007. ‘Complete correction of enzymatic deficiency and neurochemistry in the GM1-gangliosidosis mouse brain by neonatal adeno-associated virus-mediated gene delivery’, Mol Ther, 15: 30-7.

Broekman, M. L., L. A. Tierney, C. Benn, P. Chawla, J. H. Cha, and M. Sena-Esteves. 2009. ‘Mechanisms of distribution of mouse beta-galactosidase in the adult GM1-gangliosidosis brain’, Gene Ther, 16: 303-8.
Brunetti-Pierri, N., and F. Scaglia. 2008. ‘GM1 gangliosidosis: review of clinical, molecular, and therapeutic aspects’, Mol Genet Metab, 94: 391-6.
Chen, F., S. Vitry, M. Hocquemiller, N. Desmaris, J. Ausseil, and J. M. Heard. 2006. ‘alpha-L-Iduronidase transport in neurites’, Mol Genet Metab, 87: 349-58.
Chen, J. C., A. R. Luu, N. Wise, R. Angelis, V. Agrawal, L. Mangini, J. Vincelette, B. Handyside, H. J. Sterling, M. J. Lo, H. Wong, N. Galicia, G. Pacheco, J. Van Vleet, A. Giaramita, S. Fong, S. M. Roy, C. Hague, R. Lawrence, S. Bullens, T. M. Christianson, A. d'Azzo, B. E. Crawford, S. Bunting, J. H. Lebowitz, and G. Yogalingam. 2019. ‘Intracerebroventricular enzyme replacement therapy with Beta-Galactosidase reverses brain pathologies due to GM1 gangliosidosis in mice’, J Biol Chem.
Conzelmann, E., and K. Sandhoff. 1983. ‘Partial enzyme deficiencies: residual activities and the development of neurological disorders’, Dev Neurosci, 6: 58-71.
Golebiowski, D., I. M. J. van der Bom, C. S. Kwon, A. D. Miller, K. Petrosky, A. M. Bradbury, S. Maitland, A. L. Kuhn, N. Bishop, E. Curran, N. Silva, D. GuhaSarkar, S. V. Westmoreland, D. R. Martin, M. J. Gounis, W. F. Asaad, and M. Sena-Esteves. 2017. ‘Direct Intracranial Injection of AAVrh8 Encoding Monkey beta-N-Acetylhexosaminidase Causes Neurotoxicity in the Primate Brain’, Hum Gene Ther, 28: 510-22.
Gray-Edwards, H. L., X. Jiang, A. N. Randle, A. R. Taylor, T. L. Voss, A. K. Johnson, V. J. McCurdy, M. Sena-Esteves, D. S. Ory, and D. R. Martin. 2017. ‘Lipidomic Evaluation of Feline Neurologic Disease after AAV Gene Therapy’, Mol Ther Methods Clin Dev, 6: 135-42.
Gray-Edwards, H. L., A. S. Maguire, N. Salibi, L. E. Ellis, T. L. Voss, E. B. Diffie, J. Koehler, A. N. Randle, A. R. Taylor, B. L. Brunson, T. S. Denney, R. J. Beyers, A. S. Gentry, A. L. Gross, A. R. Batista, M. Sena-Esteves, and D. R. Martin. 2020. ‘7T Mill Predicts Amelioration of Neurodegeneration in the Brain after AAV Gene Therapy’, Mol Ther Methods Clin Dev, 17: 258-70.
Gray-Edwards, H. L., D. S. Regier, J. L. Shirley, A. N. Randle, N. Salibi, S. E. Thomas, Y. L. Latour, J. Johnston, G. Golas, A. S. Maguire, A. R. Taylor, D. C. Sorjonen, V. J. McCurdy, P. W. Christopherson, A. M. Bradbury, R. J. Beyers, A. K. Johnson, B. L. Brunson, N. R. Cox, H. J. Baker, T. S. Denney, M. Sena-Esteves, C. J. Tifft, and D. R. Martin. 2017. ‘Novel Biomarkers of Human GM1 Gangliosidosis Reflect the Clinical Efficacy of Gene Therapy in a Feline Model’, Mol Ther, 25: 892-903.
Hadaczek, P., J. L. Eberling, P. Pivirotto, J. Bringas, J. Forsayeth, and K. S. Bankiewicz. 2010. ‘Eight years of clinical improvement in MPTP-lesioned primates after gene therapy with AAV2-hAADC’, Mol Ther, 18: 1458-61.
Hahn, C. N., M. del Pilar Martin, M. Schroder, M. T. Vanier, Y. Hara, K. Suzuki, and A. d′Azzo. 1997. ‘Generalized CNS disease and massive GM1-ganglioside accumulation in mice defective in lysosomal acid beta-galactosidase’, Hum Mol Genet, 6: 205-11.
Hayward, C., H. C. Patel, S. G. Manohar, and A. R. Lyon. 2015. ‘Gene therapy for GM1 gangliosidosis: challenges of translational medicine’, Ann Transl Med, 3: S28.
Hinderer, C., P. Bell, N. Katz, C. H. Vite, J. P. Louboutin, E. Bote, H. Yu, Y. Zhu, M. L. Casal, J. Bagel, P. O'Donnell, P. Wang, M. E. Haskins, T. Goode, and J. M. Wilson. 2018. ‘Evaluation of Intrathecal Routes of Administration for Adeno-Associated Viral Vectors in Large Animals’, Hum Gene Ther, 29: 15-24.
Hinderer, C., P. Bell, C. H. Vite, J. P. Louboutin, R. Grant, E. Bote, H. Yu, B. Pukenas, R. Hurst, and J. M. Wilson. 2014. ‘Widespread gene transfer in the central nervous system of cynomolgus macaques following delivery of AAV9 into the cisterna magna’, Mol Ther Methods Clin Dev, 1: 14051.
Hordeaux, J., C. Hinderer, E. L. Buza, J. P. Louboutin, T. Jahan, P. Bell, J. A. Chichester, A. F. Tarantal, and J. M. Wilson. 2019. ‘Safe and Sustained Expression of Human Iduronidase After Intrathecal Administration of Adeno-Associated Virus Serotype 9 in Infant Rhesus Monkeys’, Hum Gene Ther, 30: 957-66.
Hordeaux, J., C. Hinderer, T. Goode, E. L. Buza, P. Bell, R. Calcedo, L. K. Richman, and J. M. Wilson. 2018. ‘Toxicology Study of Intra-Cisterna Magna Adeno-Associated Virus 9 Expressing Iduronate-2-Sulfatase in Rhesus Macaques’, Mol Ther Methods Clin Dev, 10: 68-78.
Hordeaux, J., C. Hinderer, T. Goode, N. Katz, E. L. Buza, P. Bell, R. Calcedo, L. K. Richman, and J. M. Wilson. 2018. ‘Toxicology Study of Intra-Cisterna Magna Adeno-Associated Virus 9 Expressing Human Alpha-L-Iduronidase in Rhesus Macaques’, Mol Ther Methods Clin Dev, 10: 79-88.
Jarnes Utz, J. R., S. Kim, K. King, R. Ziegler, L. Schema, E. S. Redtree, and C. B. Whitley. 2017. ‘Infantile gangliosidoses: Mapping a timeline of clinical changes’, Mol Genet Metab, 121: 170-79.
Kasperzyk, J. L., A. d'Azzo, F. M. Platt, J. Alroy, and T. N. Seyfried. 2005. ‘Substrate reduction reduces gangliosides in postnatal cerebrum-brainstem and cerebellum in GM1 gangliosidosis mice’, J Lipid Res, 46: 744-51.
Leinekugel, P., S. Michel, E. Conzelmann, and K. Sandhoff. 1992. ‘Quantitative correlation between the residual activity of beta-hexosaminidase A and arylsulfatase A and the severity of the resulting lysosomal storage disease’, Hum Genet, 88: 513-23.
Martin, D. R., B. A. Rigat, P. Foureman, G. S. Varadarajan, M. Hwang, B. K. Krum, B. F. Smith, J. W. Callahan, D. J. Mahuran, and H. J. Baker. 2008. ‘Molecular consequences of the pathogenic mutation in feline GM1 gangliosidosis’, Mol Genet Metab, 94: 212-21.
Matsuda, J., O. Suzuki, A. Oshima, Y. Yamamoto, A. Noguchi, K. Takimoto, M. Itoh, Y. Matsuzaki, Y. Yasuda, S. Ogawa, Y. Sakata, E. Nanba, K. Higaki, Y. Ogawa, L. Tominaga, K. Ohno, H. Iwasaki, H. Watanabe, R. O. Brady, and Y. Suzuki. 2003. ‘Chemical chaperone therapy for brain pathology in G(M1)-gangliosidosis’, Proc Natl Acad Sci USA, 100: 15912-7.
McCurdy, V. J., A. K. Johnson, H. L. Gray-Edwards, A. N. Randle, B. L. Brunson, N. E. Morrison, N. Salibi, J. A. Johnson, M. Hwang, R. J. Beyers, S. G. Leroy, S. Maitland, T. S. Denney, N. R. Cox, H. J. Baker, M. Sena-Esteves, and D. R. Martin. 2014. ‘Sustained normalization of neurological disease after intracranial gene therapy in a feline model’, Sci Transl Med, 6: 231ra48.
Mittermeyer, G., C. W. Christine, K. H. Rosenbluth, S. L. Baker, P. Starr, P. Larson, P. L. Kaplan, J. Forsayeth, M. J. Aminoff, and K. S. Bankiewicz. 2012. ‘Long-term evaluation of a phase 1 study of AADC gene therapy for Parkinson's disease’, Hum Gene Ther, 23: 377-81.
Regier, D. S., R. L. Proia, A. D'Azzo, and C. J. Tifft. 2016. ‘The GM1 and GM2 Gangliosidoses: Natural History and Progress toward Therapy’, Pediatr Endocrinol Rev, 13 Suppl 1: 663-73.
Regier, D. S., and C. J. Tifft. 2013. ‘GLB1-Related Disorders.’ in M. P. Adam, H. H. Ardinger, R. A. Pagon, S. E. Wallace, L. J. H. Bean, K. Stephens and A. Amemiya (eds.), GeneReviews((R)) (Seattle (Wash.)).
Rochette, A., M. P. Malenfant Rancourt, C. Sola, O. Prodhomme, M. Saguintaah, R. Schaub, N. Molinari, X. Capdevila, and C. Dadure. 2016. ‘Cerebrospinal fluid volume in neonates undergoing spinal anaesthesia: a descriptive magnetic resonance imaging study’, Br J Anaesth, 117: 214-9.
Sandhoff, K., and K. Harzer. 2013. ‘Gangliosides and gangliosidoses: principles of molecular and metabolic pathogenesis’, J Neurosci, 33: 10195-208.
Shield, J. P., J. Stone, and C. G. Steward. 2005. ‘Bone marrow transplantation correcting beta-galactosidase activity does not influence neurological outcome in juvenile GM1-gangliosidosis’, J Inherit Metab Dis, 28: 797-8.
Sopelsa, A. M., M. H. Severini, C. M. Da Silva, P. R. Tobo, R. Giugliani, and J. C. Coelho. 2000. ‘Characterization of beta-galactosidase in leukocytes and fibroblasts of GM1 gangliosidosis heterozygotes compared to normal subjects’, Clin Biochem, 33: 125-9.
Takaura, N., T. Yagi, M. Maeda, E. Nanba, A. Oshima, Y. Suzuki, T. Yamano, and A. Tanaka. 2003. ‘Attenuation of ganglioside GM1 accumulation in the brain of GM1 gangliosidosis mice by neonatal intravenous gene transfer’, Gene Ther, 10: 1487-93.
Tomanin, R., A. Zanetti, E. Zaccariotto, F. D'Avanzo, C. M. Bellettato, and M. Scarpa. 2012. ‘Gene therapy approaches for lysosomal storage disorders, a good model for the treatment of mendelian diseases’, Acta Paediatr.
Weismann, C. M., J. Ferreira, A. M. Keeler, Q. Su, L. Qui, S. A. Shaffer, Z. Xu, G. Gao, and M. Sena-Esteves. 2015. ‘Systemic AAV9 gene transfer in adult GM1 gangliosidosis mice reduces lysosomal storage in CNS and extends lifespan’, Hum Mol Genet, 24: 4353-64.

1. A replication deficient adeno-associated virus serotype rh. 10 (AAVrh.10)-derived vector comprising an expression cassette comprising in the following 5′ to 3′order:
a. a promoter sequence;
b. a polynucleotide sequence encoding a human β-gal or an active variant thereof; and
c. a polyadenylation (polyA) sequence.

2. The vector of claim 1, wherein the promoter sequence is derived from a CMV early enhancer/chicken beta actin (CAG) promoter sequence.

3. The vector of claim 1, wherein the polyA sequence is derived from a human growth hormone 1 sequence.

4. The vector of claim 1, wherein the expression cassette consists of, in the following 5′ to 3′order:
d. a promoter sequence derived from a CAG promoter sequence;
e. a polynucleotide sequence encoding a human β-gal or an active variant thereof; and
f. a polyA sequence derived from a human growth hormone 1 polyA sequence.

5. The vector of claim 1, wherein the expression cassette is flanked by two AAV2 internal terminal repeat (ITR) sequences, wherein one of the two AAV2 ITR sequences is located 5′ of the expression cassette and one of the two AAV2 ITR sequences is located 3′ of the expression cassette.

6. The vector of claim 5, wherein the ITR sequence located at the 5′ end of the expression cassette comprises the nucleotide sequence according to SEQ ID NO: 4 and the ITR sequence located at the 3′ end of the expression cassette comprises the nucleotide sequence according to SEQ ID NO: 5.

7. The vector of claim 2, wherein the CAG promoter sequence comprises the sequence according to SEQ ID NO: 2.

8. The vector of claim 1, wherein the polynucleotide sequence encoding a human β-gal comprises the sequence according to SEQ ID NO: 1.

9. The vector of claim 1, wherein the a polyadenylation (polyA) sequence comprises the sequence according to SEQ ID NO: 3.

10. The vector of claim 1, comprising the following in the following 5′ to 3′ order:
g. an AAV2 ITR sequence;
h. a promoter sequence derived from a CAG promoter sequence;
i. a polynucleotide sequence encoding a human β-gal or an active variant thereof;
j. a polyA sequence derived from a human growth hormone 1 polyA sequence; and
k. an AAV ITR sequence.

11. The vector of claim 1, comprising the sequence according to SEQ ID NO: 6.

12. A composition comprising the vector of claim 1 and a pharmaceutically acceptable carrier.

13. The composition of claim 12, wherein the vector is present in the composition at a concentration of about 1.0E+12 vg/mL to about 5.0E+13 vg/mL.

14. A method of treating GM1 gangliosidosis, comprising administering the vector of claim 1 to a subject in need thereof.

15. The method of claim 14, wherein the vector is administered to the cerebrospinal fluid (CSF) of the subject.

16. The method of claim 15, wherein the vector is administered to the subject via intra-cisterna magna (ICM) injection.

17. The method of claim 14, wherein the vector is administered to the subject in a volume of about 0.1 mL/kg body weight to about 1.0 mL/kg body weight.

18. (canceled)

19. The method of claim 17, wherein the vector is administered to the subject in a volume of about 0.4 mL/kg body weight.

20. The method of claim 1, wherein the vector is administered to the subject in a volume of about 1 mL to about 15 mL.

21. (canceled)

22. The method of claim 15, wherein a volume of cerebrospinal fluid (CSF) is removed prior to administration of the vector.

23. (canceled)

24. The method of claim 14, wherein the subject is administered a dose of the vector of between about 1.0E+12 vg/kg body weight to about 1.0E+13 vg/kg body weight.

25. The method of claim 24, wherein the subject is administered a dose of the vector of about 8.0E+12 vg/kg body weight.

26. The method of claim 24, wherein the subject is administered a dose of the vector of about 5.0E+11 vg/mL of CSF to about 5.0E+12 vg/mL of CSF.

27. The method of claim 24, wherein the subject is administered a dose of the vector of about 1.8E+12 vg/mL of CSF.

28. The method of claim 14, wherein the method further comprises administering an immunosuppressive regimen to the subject.

29. The method of claim 28, wherein the immunosuppressive regimen comprises tacrolimus, mycophenolate mofetil, and prednisone.

30.-32. (canceled)

33. A composition according to claim 12 for use as a medicament in the treatment of GM1 gangliosidosis.

34. The composition of claim 33, for administration to the cerebrospinal fluid (CSF) of the subject.

35. The composition of claim 34, wherein the vector is for administration via intra-cisterna magna (ICM) injection.

36. A kit comprising a vector according to claim 1 and instructions for use thereof.
